SEQUENCE LISTING (13 sequences)

SEQ ID NO: 1 - DNA, 5 bp, Artificial
  Synthetic sequence
ccctt

SEQ ID NO: 2 - DNA, 53 bp, Artificial
  Synthetic sequence
tgacgcccgaagccaagtgcgggacggcttctccagcttggccccttatgggt

SEQ ID NO: 3 - DNA, 57 bp, Artificial
  Synthetic sequence
tcgttcccataaggggccaagctggagaagccgtcccgcacttggcttcgggcgtca

SEQ ID NO: 4 - DNA, 39 bp, Artificial
  Synthetic sequence
atgggaacgagcagaccgaccgctagacagctccgtgga

SEQ ID NO: 5 - DNA, 29 bp, Artificial
  Synthetic sequence
tccacggagctgtctagcggtcggtctgc

SEQ ID NO: 6 - DNA, 47 bp, Artificial
  Synthetic sequence
tgacgcccgaagccaagtgcgggacggcttctccagcttggcccctt

SEQ ID NO: 7 - DNA, 86 bp, Artificial
  Synthetic sequence
tgacgcccgaagccaagtgcgggacggcttctccagcttggccccttatgggaacgagcagaccgaccgctagacagctccgtgga

SEQ ID NO: 8 - DNA, 86 bp, Artificial
  Synthetic sequence
tccacggagctgtctagcggtcggtctgctcgttcccataaggggccaagctggagaagccgtcccgcacttggcttcgggcgtca

SEQ ID NO: 9 - RNA, 190 nt, Artificial
  Synthetic sequence
gaauuggagcuccaccgcgguggcggccgcucuagaacuaguggaucccccgggcugcaggaauucgauaucaagcuuaucgauaccgucgaccucgagggggggcccgguacccagcuuuuguucccuuuagugaggguuaauugcgcgcuuggcguaaucauggucauagcuguuuccugugugaaau

SEQ ID NO: 10 - DNA, 19 bp, Artificial
  Synthetic sequence
tccacggagctgtctagcg

SEQ ID NO: 11 - DNA, 19 bp, Artificial
  Synthetic sequence
gtgaaccgaagcccgcagt

SEQ ID NO: 12 - DNA, 6 bp, Artificial
  Synthetic sequence
atgggt

SEQ ID NO: 13 - DNA, 11 bp, Artificial
  Synthetic sequence
cccttnnnnnn

(19) United States
(12) Patent Application Publication
(10) Pub. No.: US 2009/0075259 A1
(43) Pub. Date: Mar. 19, 2009

(54) Analyte detection via antibody-associated enzyme assay

(76) Inventors: Carsten-Peter Carstens, San Diego, CA (US); Bernd Buehler, San Diego, CA (US)

Correspondence Address: AGILENT TECHOLOGIES INC, P.O BOX 7599, BLDG E , LEGAL, LOVELAND, CO, 80537-0599, US

(21) Appl. No.: 11/897,886

(22) Filed: Aug. 31, 2007

(51) Int. Cl. C12Q 1/68

(52) U.S. Cl. 435/6

(57) ABSTRACT

The present invention provides methods, kits, and compositions for the detection of an analyte. The invention is particularly suited for the detection and quantification of an analyte in a sample. In the methods of the invention a complex is formed between an analyte specific binding agent and an analyte. The analyte specific agents are coupled to an enzyme possessing an activity that produces a PCR template indicative of the presence of the analyte. Amplification and detection of the PCR template yields a sensitive and quantitative measurement of analyte concentration.

### BACKGROUND OF THE INVENTION

The development of immunoassays and advances in nucleic acid detection have advanced the detection of analytes in biological samples. The enzyme-linked immunosorbent assay (ELISA) allows for the high throughput screening of samples for the presence of proteins in samples. The presence of the analyte is frequently detected by the use of an enzymatic, colorimetric assay based on alkaline phosphatase or horseradish peroxidase. This limits the sensitivity and the range of the assay depending on the range of detection of colorimetric changes of the enzyme substrate. This requires either initial screening to determine an approximate amount of the analyte in the serum, or the use of a large series of dilutions to insure that a sample is tested within the detection range of the specific methods and reagents used.

To overcome these limitations, nucleic acid based detection methods for use in conjunction with enzyme-based detection of analytes in samples have been developed. Such methods are sometimes referred to as immuno-PCR. Assays and methods are described, for example in Niemeyer et al (Nuc. Acid Res. 31:e90, 2003), U.S. Patent Publication Nos. 2002/0064779 and 2005/0003361; U.S. Pat. No. 6,511,809; and PCT Publications WO2005/019470 and WO2007/044903.

### SUMMARY OF THE INVENTION

As described below, the present invention relates to methods, kits, and compositions for detection and quantitation of an analyte in a sample using an analyte-specific binding agent. The analyte specific binding agent includes an analyte-specific binding molecule attached to an enzyme. The analyte-specific binding agent is incubated with a sample under conditions for binding of the analyte-specific binding agent to the analyte for detection of an analyte corresponding to the specific binding agent used. The bound analyte-specific binding agent is incubated with one or more polynucleotides, depending on the enzyme in the analyte-specific binding agent, under conditions to permit the enzyme to generate a template for a nucleic acid amplification reaction. The template is preferably a template for a polymerase chain reaction (PCR) that can be used to produce an amplification product. Detection of the presence of the amplification product is indicative of the presence of the analyte in the sample. Quantitative amplification reactions can be performed to quantify the amount of analyte in the sample.

In an aspect, the invention relates to methods for detection of an analyte in a sample by contacting an analyte-specific binding agent with a sample that may contain the analyte under conditions to permit binding. The analyte-specific binding agent comprises an analyte-specific binding molecule attached to a ligase. The ligase activity can be derived from either a topoisomerase or a ligase. The bound analyte-specific binding agent is further incubated with a substrate for a ligase activity. The substrate includes a first and a second ends of at least one double stranded DNA molecule, wherein the ends are cohesive compatible ends that can anneal and be ligated to each other under conditions to permit ligation (e.g., in a ligation mixture) to form an amplification template. The ends can be provided by at least one double stranded DNA molecule, but can be provided by at least two distinct double stranded DNA molecules. At least a portion of the ligation mixture is incubated in an amplification reaction mixture including at least two oligonucleotide primers that hybridize to opposite strands of the template in an orientation to allow an amplification product to be produced in the presence of at least one dNTP, preferably all four dNTPs, and a polymerase. The amplification product is detected during and/or after the amplification reaction to determine if analyte is present in the sample. The amplification product can be detected during the amplification by qPCR or other methods.

The invention further relates to methods for detection of an analyte in a sample by incubating an analyte-specific binding agent with a sample under conditions to permit binding of the analyte, wherein the analyte-specific binding agent comprises an analyte-specific binding molecule attached to a reverse transcriptase. The bound analyte-specific binding agent is further incubated with an RNA molecule that can act as a substrate in a reverse transcription reaction mixture to form a cDNA that can, in turn, act as template for nucleic acid amplification. At least a portion of the reverse transcription reaction mixture is incubated in an amplification reaction mixture including preferably at least two oligonucleotide primers. Primers are designed such that one that can hybridize to the cDNA to permit the polymerization of a complementary strand, and one can hybridize to the complementary strand of the cDNA. The primers are designed to hybridize to the cDNA and the complementary strand in an orientation to allow an amplification product to be produced in the presence of at least one dNTP, preferably all four dNTPs, and a polymerase. The amplification product is detected during and/or after the amplification reaction to determine if analyte is present in the sample. The amplification product can be detected during the amplification by qPCR or other methods.

In an aspect, the invention relates to kits for practicing the methods of the invention. Kits include an analyte-specific binding agent comprising an analyte-specific binding molecule attached to an enzyme; one or more polynucleotide substrates for the enzyme moiety, and packing material therefor. The kit can further include one or more reagents for the amplification of the nucleic acid step such as reverse transcriptase, reagents for PCR, particularly qPCR, and primers. In lieu of an analyte specific binding agent, the kit can include an enzyme having a group for attachment to an analyte-specific binding moiety, such as an antibody. For example, an enzyme attached to protein A, protein G, or protein L can be mixed with an antibody by the end user to produce an analyte-specific binding agent. Alternatively, the enzyme can be attached to streptavidin or avidin and mixed with a biotinylated antibody obtained from another source (e.g., commercial source or generated in the laboratory). Other methods for attachment of analyte specific binding molecules and enzymes to each other are discussed below.

In an aspect, the invention includes analyte-specific binding agents including an analyte-specific binding moiety and an enzyme. Analyte-specific binding agents can include an analyte specific binding molecules selected from the group consisting of monoclonal antibody, polyclonal antibody, lectin, cell surface receptor, receptor ligand, peptide, carbohydrate, aptamer, biotin, streptavidin, avidin, protein A, protein G, and protein L; and any binding fragments thereof. Enzymes can include topoisomerase, ligase, and reverse transcriptase.

### DEFINITIONS

As used herein, the term “amplification” or “synthesis,” when applied to a nucleic acid sequence, refers to a process whereby one or more copies of a particular nucleic acid sequence is generated from a template nucleic acid. Amplification, as used herein, is meant to include a single replication/copying of a nucleic acid sequence such that by a primer extension reaction. However, generally amplification is carried out using a polymerase chain reaction (PCR) or ligase chain reaction (LCR) technologies well known in the art (Dieffenbach, C. W. and G. S. Dveksler (1995) PCR Primer, a Laboratory Manual, Cold Spring Harbor Press, Plainview, N.Y.). In addition, the methods of the invention may be practiced using Strand Displacement Amplification (SDA), Rolling Circle Amplification (RCA), Transcription Mediated Amplification (TMA) or Ligase Chain Reaction (LCR). Amplification of signal may be generated in a homogeneous, closed tube environment, using Real-Time amplification. Instrumentation suitable for Real-Time amplification includes the Stratagene Mx3005P, ABI PRISM TaqMan system, Roche LightCycler, Idaho Technologies RapidCycler, Bio-Rad icycler and Cepheid SmartCycler.

As used herein, “amplification product” refers to the polynucleotide produced by a polymerization reaction using a thermostable or non-thermostable DNA polymerase. In a preferred embodiment, the polymerization reaction is a polymerase chain reaction. The amplification product can be detected by qualitative or semi-quantitative methods, for example, by gel electrophoresis and staining or dot blot, using samples from an amplification reaction obtained at one or more time points during and/or after the amplification reaction. The amplification product can be detected throughout the amplification reaction by the use of quantitative PCR methods by fluorescent monitoring using any of a number of commercially available reagents such as those noted above.

In an embodiment, exponential amplification can be achieved using a single-stranded polynucleotide template and a single primer. This is achieved by designing the polynucleotide template sequence to contain a primer binding sequence at one end of the single stranded target and a complement sequence of the primer binding site at the opposite end of the target strand. Annealing and extension of the primer results in the formation of a complementary target strand containing the identical primer binding sites. In this way both the (+) and (−) strands of the resulting double stranded target contain an identical primer site at opposite ends of the target duplex, and the same primer used in combination with the polymerase and target nucleic acid promotes replication of both + and − target strands.

As used herein the term “analyte” refers to a substance to be detected or assayed by the method of the present invention. Typical analytes may include, but are not limited to proteins, peptides, cell surface receptors, receptor ligands, nucleic acids, carbohydrates, molecules, cells, microorganisms and fragments thereof, or any substance for which an analyte-specific binding molecule, e.g., antibodies, can be developed.

As used herein the terms “analyte-specific binding agent”, refers to a molecule having an analyte specific binding molecule attached or coupled to an at least an active portion of enzyme. These portions can be attached, for example, by expression of the two portions as a single fusion protein, with or without intervening sequences not native to either protein. Coding sequences for generic antibody-binding ligands such as protein A, G, or L can be fused to the coding sequence of the enzyme and mixed with the antibody to attach the enzyme to the analyte-binding molecule. High affinity binding partners such as biotin and streptavidin can also be used. Biotin can be linked to either portions of the analyte-specific binding agent, and avidin or streptavidin can be linked to the other. The analyte-specific binding molecule, e.g., anti-analyte mAb, can be attached to enzyme via a cross-linker to form an analyte-specific binding agent. Any cross-linking chemistry known in art for conjugating proteins can be used in conjunction with the present invention.

The binding moiety is operatively coupled to the enzymatic moiety such that the binding molecule does not substantially interfere with the activity of the enzyme, and vice versa. The coupling does reduces the activity of the enzyme by less than 70%, 60%, 50%, 40% or 30%, preferably less than 25%, 20%, 15%, or 10%, more preferably less than 5%, 3%, 2%, or 1%. Similarly, the coupling reduces the affinity of the binding moiety less than 70%, 60%, 50%, 40% or 30%, preferably less than 25%, 20%, 15%, or 10%, more preferably less than 5%, 3%, 2%, or 1%. The invention is not limited by the specific structure or method of attachment of the moieties of the analyte-specific binding agent. Typically the analyte-specific binding molecule and the enzyme are present at about a 1:1 ratio; however, other ratios are possible provided that the function of the various portions is not substantially inhibited by the presence of the other moieties.

As used herein, the term “annealing” means permitting oligonucleotide primers to hybridize to complementary, typically complementary cohesive ends or template nucleic acid strands. Conditions for primer annealing vary with the length and sequence of the primer and are based upon calculated Tₘ for the primer. As used herein, “under conditions to permit annealing” is understood to be in a reaction having appropriate conditions including, but not limited to, appropriate salt, cation, buffer, and complementary nucleic acid concentrations; and appropriate temperature such that formation of double stranded nucleic acid molecules is possible. As used herein, the double stranded nucleic acid molecules are preferably formed by two separate nucleic acid molecules. Generally, an annealing step in an amplification regimen involves reducing the temperature following the strand separation step to a temperature based on the calculated Tₘ for the primer sequence, for a time sufficient to permit such annealing.

As used herein, the term “antibody” refers to an immunoglobulin protein that is capable of binding an antigen, e.g., analyte. Antibody includes any portion of an antibody that retains the ability to bind to the epitope recognized by the full-length antibody, generally termed “epitope-binding fragments.” Examples of antibody fragments preferably include, but are not limited to, Fab, Fab′, and F(ab′)₂, Fd, single-chain Fvs (scFv), single-chain antibodies, disulfide-linked Fvs (sdFv) and fragments comprising either a VL or VH domain. Epitope-binding fragments, including single-chain antibodies, may comprise the variable region(s) alone or in combination with the entirety or a portion of the following: hinge region, C_{H}¹, C_{H}², and C_{H}³ domains.

As used herein the term “analyte-specific binding molecule” refers to a molecule or portion of a molecule that stably binds an analyte. Binding molecules include, but are not limited, to monoclonal antibody, polyclonal antibody, aptamer, cell surface receptor, receptor ligand, biotin, streptavidin, avidin, and protein A, G, and L. Binding molecules can also be binding fragments of the binding moieties listed, e.g., antibody fragments as listed above. The binding molecules is directly or indirectly coupled to an enzyme to form the analyte-specific binding agent.

As used herein, a “bound analyte-specific binding agent” is an analyte-specific binding agent bound to its corresponding analyte.

As used herein, “Cₜ” refers to the cycle number at which the signal generated from a quantitative amplification reaction first rises above a “threshold”, i.e., where there is the first reliable detection of amplification of a target nucleic acid sequence. “Reliable” means that the signal reflects a detectable level of amplified product during amplification. Cₜ generally correlates with starting quantity of an unknown amount of a target nucleic acid, i.e., lower amounts of target result in later Cₜ. Cₜ is linked to the initial copy number or concentration of starting nucleic acid.

By “capture molecule” is meant a specific or non-specific agent on a solid support to bind the analyte. The capture molecule can be an antibody that binds the analyte specifically. The capture molecule can also be a nucleic acid sequence, single or double stranded, DNA or RNA that is bound by the analyte. Alternatively, the capture molecule can be poly-lysine, silane, collagen, or other non-specific agent to capture the analyte on the solid support.

As used herein, the “catalytic portion” of a ligase, polymerase, topoisomerase, or other enzyme is the portion of the enzyme required to promote the enzymatic reaction required for the methods of the invention. Structures of such enzymes are known, and structure-function relationships between various amino acids and domains and enzymatic activity are well understood (see, e.g., on topoisomerases Champoux et al., Annu. Rev. Biochem. 70:369-413, 1991; and on polymerases, Braithwaite and Ito, Nuc. Acids Res. 19:4045, 1991, and Brathwaite and Ito, Nucleic Acids Res. 21: 787, 1993; all of which are incorporated herein by reference). Enzymes containing truncations and mutations that do not substantially alter the specific catalytic activity of the enzymes required for the methods of the invention are included in the scope of the invention.

As used herein, the term “cDNA” refers to complementary or copy polynucleotide produced from an RNA template by the action of RNA-dependent DNA polymerase (e.g., reverse transcriptase). A “cDNA clone” refers to a duplex DNA sequence complementary to an RNA molecule of interest, carried in a cloning vector.

As used herein, “cleavage” refers to the cutting, typically enzymatic cutting, of one or both strands of a single-stranded or double-stranded polynucleotide.

As used herein, the term “cleavage product” is a polynucleotide fragment that is released into solution after cutting of one or both strands of the polynucleotide. In some embodiments, the cleavage product is an oligonucleotide cleaved by a topoisomerase. In another embodiment, the cleavage product is an oligonucleotide cleaved by a restriction enzyme. A cleavage product may be a short, single stranded portion previously hybridized to a complementary strand. Alternatively, a cleavage product may be double stranded.

As used herein, a “cleavage site” refers to a polynucleotide structure or sequence that is capable of being cleaved by a cleavage agent. Cleavage sites include, but are not limited to, topoisomerase enzyme recognition sites, restriction enzyme sites, ribozyme sites, nickase sites, DNAzyme sites and nuclease cleavage sites. For example, the specific cleavage recognition site for vaccinia-virus based topoisomerase and MCV topoisomerase is CCCTT. The cleavage occurs after the final T. Cleavage sites for restriction enzymes are well known and can be found in any of a number of catalogs for molecular biology reagents.

As used herein, “compatible cohesive ends” are typically short, (less than about 20, less than about 15, less than about 10 nucleotides in length) single-stranded ends of double stranded DNA molecules that are capable of hybridizing under conditions that permit ligation to form a substrate for a ligase. By increasing the length of the annealed portion of the cohesive ends, the temperature at which the strands are stably annealed increases, allowing for use of the ligases at higher temperatures (e.g., 37° C.). Such substrates include a gap in the nucleotide backbone, but no gaps in the nucleotide pairing. Ligation by topoisomerase requires the presence of a free 5′-OH at the 5′-end of the acceptor molecules, and ligation by conventional ligases require a 5′phosphate group.

As used herein, “complementary” refers a capacity for precise pairing of purine and pyrimidine bases between strands of DNA, and sometimes RNA, such that the structure of one strand determines the other. A first polynucleotide is said to be “fully complementary” or “completely complementary” to a second polynucleotide strand if each and every nucleotide of the first polynucleotide forms basepairs with nucleotides within the complementary region of the second polynucleotide. A first polynucleotide is not completely complementary (i.e., it is partially complementary) to the second polynucleotide if one nucleotide in the first polynucleotide does not base pair with the corresponding nucleotide in the second polynucleotide. For example, two polynucleotides may be 60%, 65%, 70%, 75%, 80%, 85%, 90%, 95%, 98%, or 99% complementary. The percent complementarity can be determined, for example, by dividing the number of complementary bases by the total length of the double stranded portion or the length of the shorter strand of the polynucleotide. The degree of complementarity between polynucleotide strands has significant effects on the efficiency and strength of annealing or hybridization between polynucleotide strands. This is of particular importance in amplification reactions, which depend upon binding between polynucleotide strands. An oligonucleotide need not be 100% complementary to a template to permit amplification. Mismatches between an oligonucleotide and a template are tolerated more near the 5′-end of the oligonucleotide than the 3′-end of the oligonucleotide in extension reactions. Typically, a mismatch at the terminal 3′-nucleotide of the oligonucleotide will inhibit extension by a polymerase.

As used herein, “conditions to permit” binding, amplification or formation of an amplification product, ligation, hybridization, and the like are understood to be in the presence of the necessary reagents such as salts, buffer, nucleotides, enzyme, divalent cations, ATP, and appropriate conditions, of pH and temperature for appropriate amounts of time. Conditions that permit activity of a particular enzyme are typically provided by the enzyme manufacturer. Conditions that permit binding of antibodies can be found, for example, in Harlow and Lane (Eds.), Antibodies: A Laboratory Manual. Cold Spring Harbor Laboratory Press, Cold Spring Harbor, N.Y., ©1988 (incorporated herein by reference). Conditions that permit amplification, formation of an amplification product, and hybridization of oligonucleotides can be found, for example, in Chen and Janes (Eds.), PCR Cloning Protocols (Methods in Molecular Biology). Humana Press, Inc., Totowa, N.J., ©2002 (incorporated herein by reference).

As used herein, “coupled” refers to the association of two molecules though covalently and non-covalent interactions, e.g., by hydrogen, ionic, or Van-der-Waals bonds. Such bonds may be formed between at least two of the same or different atoms or ions as a result of redistribution of electron densities of those atoms or ions. For example, an enzyme may be coupled to an antibody as an antibody-enzyme fusion protein, via binding through a streptavidin-biotin interaction or through binding via an Fc protein A/G/L interaction (e.g., polymerase is coupled to protein A/G which in turn binds the Fc region of the antibody).

As used herein, “detecting”, “detection” and the like are understood that an assay was performed for a specific analyte in a sample. The amount of analyte detected in the sample can be none or below the level of detection of the assay.

As used herein, “dNTP” is understood as deoxynucleotide triphosphate which includes the natural or “standard” dNTPs, dATP, dCTP, dGTP, and TTP. As used herein, dNTP also include natural and non-natural nucleotide analogs, such as fluorescently or otherwise chemically labeled nucleotides.

As used herein, “double-stranded DNA” is understood to mean DNA that has at least a portion that is annealed to a complementary strand or segment of DNA. Double stranded DNA can be comprised of two separate strands or can be a single polynucleotide with self-complementary sequences (e.g., a hairpin structure). A double-stranded DNA molecule or polynucleotide can include single stranded portions.

As used herein, an “enzyme” includes at least the catalytic portion of an enzyme, such as topoisomerase, ligase, reverse transcriptase, that can be attached to an analyte specific binding moiety. The enzyme portion can exist independently of the analyte specific binding moiety.

As used herein, a “fusion polypeptide” refers to a polypeptide comprising two or more polypeptides that are linked (coupled) in frame to each other. As used herein, the term “linked” or “fused” means the linking together of two or more segments of a polypeptide or nucleic acid to form a fusion molecule that encodes two or more polypeptides linked in frame to each other. The two or more polypeptides may be linked directly or via a linker sequence.

As used herein, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds.

As used herein, “isolated” or “purified” when used in reference to a polynucleotide means that a naturally occurring sequence has been dispensed from its normal cellular (e.g., chromosomal) environment or is synthesized in a non-natural environment (e.g., artificially synthesized). Thus, an “isolated” or “purified” sequence can be in a cell-free solution or placed in a different cellular environment. The term “purified” does not imply that the sequence is the only nucleotide sequence present, but that it is essentially free (about 90-95%, up to 99-100% pure) of non-nucleotide or polynucleotide material naturally associated with it, and thus is distinguished from isolated chromosomes.

As use herein, a “ligase” is at least a portion of a ligase or topoisomerase enzyme that is capable of catalyzing the joining the adjacent ends of DNA strands in an enzyme appropriate ligase substrate.

As used herein, “melting temperature” or “Tₘ” is understood as a temperature value that is related to the affinity of two complementary nucleic acid molecules for each other. A Tₘ can be readily predicted by one of skill in the art using any of a number of widely available algorithms (e.g., Oligo™, Primer Design, and programs available on the internet, including Primer3 and Oligo Calculator). For most amplification regimens the annealing temperature is elected to be about 5° C. below the predicted Tₘ, although temperatures closer to and above the Tₘ (e.g., between 1° C. and 5° C. below the predicted Tₘ or between 1° C. and 5° C. above the predicted Tₘ) can be used, as can temperatures more than 5° C. below or above the predicted Tₘ (e.g., 6° C. below, 8° C. below, 10° C. below or lower and 6° C. above, 8° C. above, or 10° C. above). Generally, the closer the annealing temperature is to the Tₘ, the more specific is the annealing. Time of primer annealing depends largely upon the volume of the reaction, with larger volumes requiring longer times, but also depends upon primer and template concentrations, with higher relative concentrations of primer to template requiring less time than lower. Depending upon volume and relative primer/template concentration, primer annealing steps in an amplification regimen can be on the order of 1 second to 5 minutes, but will generally be between 10 seconds and 2 minutes.

As used herein, the term “moiety” or “portion” is understood as one of the active domains into which something, such as an analyte-specific detection agent, is divided. A moiety or portion may exist independently of the analyte specific detection agent.

As used herein, the term “oligonucleotide” or “polynucleotide” refers polydeoxyribonucleotides (containing 2-deoxy-D-ribose), to polyribonucleotides (containing D-ribose) and to any polynucleotide which is an N-glycoside of a purine or pyrimidine base, or modified purine or pyrimidine base. An oligonucleotide may hybridize to other oligonucleotide or may self-hybridize, e.g., hairpin structure. An oligonucleotide includes, without limitation, single- and double-stranded oligonucleotides.

As used herein, “plurality” is understood to mean more than one, typically at least two. As used herein, a “polymerase” is an enzyme that catalyzes the polymerization of nucleotides in a template dependent manner. Polymerases can use DNA or RNA as a template. Polymerases can be thermostable or non-thermostable. Generally, the enzyme will initiate synthesis at the 3′-end of the primer annealed to a nucleic acid template sequence, and will proceed toward the 5′ end of the template strand. “DNA polymerase” catalyzes the polymerization of deoxyribonucleotides. Known DNA polymerases include, for example, Pyrococcus furiosus (Pfu) DNA polymerase (Lundberg et al., 1991, Gene, 108:1), E. coli DNA polymerase I (Lecomte and Doubleday, 1983, Nucleic Acids Res. 11:7505), T7 DNA polymerase (Nordstrom et al., 1981, J. Biol. Chem. 256:3112), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent DNA polymerase, Cariello et al., 1991, Nucleic Acids Res, 19: 4193), 9° Nm DNA polymerase (discontinued product from New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), Pyrococcus kodakaraensis (KOD) DNA polymerase (Takagi et al., 1997, Appl. Environ. Microbiol. 63:4504), JDF-3 DNA polymerase (Patent application WO 0132887), and Pyrococcus GB-D (PGB-D) DNA polymerase (Juncosa-Ginesta et al., 1994, Biotechniques, 16:820). The polymerase activity of any of the above enzyme can be determined by means well known in the art. One unit of DNA polymerase activity, according to the subject invention, is defined as the amount of enzyme which catalyzes the incorporation of 10 nmoles of total dNTPs into polymeric form in 30 minutes at optimal temperature (e.g., 72° C. for Pfu DNA polymerase). No thermostable DNA polymerases include, but are not limited to, T3 DNA polymerase, T4 DNA polymerase, T5 DNA polymerase, T7 DNA polymerase, Klenow fragment, E. coli DNA polymerase I, and Φ29 DNA polymerase.

As used herein, a “polynucleotide” generally refers to any polyribonucleotide or poly-deoxyribonucleotide, which can be unmodified RNA or DNA, or modified RNA or DNA. “Polynucleotides” include, without limitation, single- and double-stranded polynucleotides. As used herein, the term “polynucleotide(s)” also includes DNAs or RNAs, that contain one or more modified bases. Thus, DNAs or RNAs with backbones modified for stability or for other reasons are “polynucleotides”. The term “polynucleotides” as it is used herein embraces such chemically, enzymatically or metabolically modified forms of polynucleotides, as well as the chemical forms of DNA and RNA characteristic of viruses and cells, including for example, simple and complex cells. A polynucleotide useful for the methods herein can be an isolated or purified polynucleotide or it can be an amplified polynucleotide in an amplification reaction.

As used herein, “polynucleotide substrate molecule(s) for an enzyme” is understood as one or more DNA or RNA molecules that can be acted on catalytically by an enzyme to produce an intermediate or product. A “polynucleotide substrate for amplification or PCR” is understood as a single or double stranded DNA polynucleotide of sufficient length to permit binding of two specific oligonucleotide primers (one to a first strand, and one to a second or complementary strand synthesized using the first strand as a template). For example, an RNA molecule can be a substrate for reverse transcriptase. Two double stranded DNA molecules with compatible ends can be a substrate for ligase. A double-stranded DNA molecule including a 5′-CCCTT-3′ (SEQ ID NO: 1) sequence can be a substrate for vaccinia virus DNA topoisomerase I. A double stranded DNA molecule of sufficient length and appropriate sequence to allow for the specific binding of two primers can be the substrate for nucleic acid amplification by PCR.

The term “primer” may refer to more than one primer and refers to an oligonucleotide, whether occurring naturally, as in a purified restriction digest, or produced synthetically, which is capable of acting as a point of initiation of synthesis along a complementary strand when placed under conditions in which synthesis of a primer extension product which is complementary to a nucleic acid strand is catalyzed. Such conditions include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.), and at a suitable temperature. The primer is preferably single-stranded for maximum efficiency in amplification.

Oligonucleotide primers useful according to the invention are single stranded DNA or RNA molecules that are hybridizable to a template nucleic acid sequence and prime enzymatic synthesis of a second nucleic acid strand. The primer is complementary to a portion of a target molecule. It is contemplated that oligonucleotide primers according to the invention are prepared by synthetic methods, either chemical or enzymatic. Alternatively, such a molecule or a fragment thereof is naturally-occurring, and is isolated from its natural source or purchased from a commercial supplier. Oligonucleotide primers and probes are 5 to 100 nucleotides in length, ideally from 17 to 40 nucleotides, although primers and probes of different length are of use. Primers for amplification are preferably about 17 to 25 nucleotides. Primers useful according to the invention are also designed to have a particular melting temperature (Tₘ) by the method of melting temperature estimation. Commercial programs, including Oligo™, Primer Design and programs available on the internet, including Primer3 and Oligo Calculator can be used to calculate a Tₘ of a nucleic acid sequence useful according to the invention. Preferred, Tₘ's of a primer will depend on the particular embodiment of the invention that is being practiced. The oligonucleotides of the invention include polynucleotide templates (modified or non-modified) and primers. The polynucleotide templates can be prepared with lengths ranging in length from at least 10 bases in length, typically at least 20 bases in length, for example, at least 30, 40, 50, 60, 70, 80, 90 or 100 bases in length. While the oligonucleotide can be large nucleic acid fragments, it is generally limited to nucleic acids of 500 bases or less.

The oligonucleotides of the invention may be free in solution or conjugated to a binding molecule. Oligonucleotides that are conjugated to a binding moiety will generally have a chemically active group (such as, primary amine group) at any point in its stretch of nucleic acids, which allows it to be conjugated.

As used herein a “reaction mixture” is a combination of reagents, typically including, but not limited to, salt(s), buffer(s), nucleic acid(s), and enzyme(s). A reaction mixture is typically exposed to conditions under which the desired reaction can occur. Conditions under which a reaction can occur are frequently provided in manufacturer's instructions provided with at least some reagents, for example enzymes.

As used herein, the term “restriction enzyme” refers to an enzyme that cuts double-stranded DNA at or near a specific nucleotide sequence. The specificities of numerous restriction enzymes are well known in the art. Various restriction enzymes are commercially available and their reaction conditions, cofactors, and other requirements as established by the enzyme suppliers are well known.

As used herein, a “reverse transcriptase” is an RNA-dependent DNA polymerase, including MMLV and AMV reverse transcriptases. A number of reverses transcriptases for use at different temperatures are commercially available including AffinityScript™, AccuScript®, and StrataScript® (all from Stratagene).

As used herein, the term “sample” refers to a biological material that is isolated from its natural environment and is suspected of, or possibly containing an analyte. A “sample” according to the methods disclosed herein can contain a purified or isolated analyte, or it can comprise a biological sample such as a tissue sample, a biological fluid sample, or a cell sample suspected of containing an analyte. A biological fluid includes blood, plasma, serum, sputum, urine, cerebrospinal fluid, lavages, and leukophoresis samples. A sample can comprise any plant, animal, bacterial or viral material suspected of containing an analyte.

As used herein, a “solid support” or “solid surface” refers to any structure that provides a support for the capture molecule. Suitable solid supports include polystyrene, derivatized polystyrene, a membrane, such as nitrocellulose, PVDF or nylon, a latex bead, a glass bead, a silica bead, paramagnetic or latex microsphere, or microtiter well. As a further example, the solid support may be a modified microtiter plate, such as a Top Yield™ plate, which allows for covalent attachment of a capture molecule, such as an antibody, to the plate. When the solid support is a material such as a bead, paramagnetic microsphere or latex microsphere, the solid support may be contained in an open container, such as a multi-well tissue culture dish, or in a sealed container, such as a screw-top tube, both of which are commonly used in laboratories.

By the terms “specifically binding” and “specific binding” as used herein is meant that an antibody or other binding molecule, especially a receptor of the invention, binds to a target such as an antigen, ligand or analyte, with greater affinity than it binds to other molecules under the specified conditions of the present invention. Antibodies or antibody fragments, as known in the art, are polypeptide molecules that contain regions that can bind other molecules, such as antigens. In various embodiments of the invention, “specifically binding” may mean that an antibody or other biological molecule, binds to a target molecule with at least about an affinity of 10⁻⁶-10⁻¹⁰/M, more preferably they will have an affinity of at least 10⁻⁸/M, most preferably they will have an affinity at least 10⁻⁹/M.

As used herein, a “substrate for ligase” is understood herein to be a pair of double stranded nucleic acid molecules, or the two ends of one double stranded nucleic acid molecule, having compatible cohesive ends such that annealing of the cohesive ends to each other results in a double stranded nucleic acid molecule with a break in the backbone of each strand, without missing any nucleotides. The nucleic acid strands are fully hybridized to the complementary strand at and around the site of the break on at least one strand, for at least about 3, 5, 7, or 10 consecutive complementary nucleotides at and around the break. Both strands are DNA molecules, or one of the strands is a DNA molecule and the other strand is an RNA molecule. The ends of the strands at the point of the break have the appropriate terminal functional groups to allow for ligation by a ligase or a topoisomerase. Ligases require a 5′ phosphate group at the 5′ end of the acceptor molecule, whereas topoisomerases require a free 5′-OH at the 5′ end of the acceptor molecules. Ligases can be thermostable or non-thermostable ligases. Non-thermostable ligases can be inactivated by exposure to elevated temperature, for example, the denaturing step of a polymerase chain reaction.

As used herein, a “substrate for polymerase” is understood herein to be a single stranded nucleic acid, either DNA, with a portion of double stranded sequence wherein the 3′ end of the first strand of the double stranded portion is fully complementary to the sequence to which is annealed, and the second strand extends beyond the 3′ end of the first strand in the direction in which the 3′ end would extend. The 3′ end further includes a 3′-hydroxyl group to allow for extension of the 3′ end. Such a DNA template is also a “PCR template”. Alternatively, the nucleic acid can be an RNA strand from which a cDNA can be generated which, in turn, can act as a PCR template. Such an RNA strand is also a “substrate for reverse transcriptase.”

As used herein, a “thermostable polymerase” is understood as an enzyme that is stable to heat, is heat resistant and catalyzes (facilitates) combination of the nucleotides in the proper manner to form the primer extension products that are complementary to each nucleic acid strand. A thermostable polymerase does not become irreversible denatured (inactivated) when subjected to the elevated temperatures for the time necessary to effect denaturation of double-stranded nucleic acids. Irreversible denaturation for purposes herein refers to permanent and complete loss of enzymatic activity. The heating conditions necessary for nucleic acid denaturation will depend, e.g., on the buffer salt concentration and composition and the length and nucleotide composition of the nucleic acids being denatured, but typically range from about 90 to about 105° C. for a time depending mainly on the temperature and the nucleic acid length, typically about 30 seconds to four minutes. Higher temperatures may be tolerated as the buffer salt concentration and/or GC composition of the nucleic acid is increased. Preferably, the enzyme will not become irreversible denatured at about 90-100° C. “Non-thermostable polymerase” is understood to mean a polymerase that becomes irreversibly denatured under conditions tolerated by thermostable polymerases. Both thermostable and non-thermostable polymerases are widely available from a number of commercial suppliers.

As used herein, a “topoisomerase” refers to at least the catalytic portion of an enzyme that can mediate the cleavage and ligation of DNA. Some topoisomerases catalyze cleavage of one strand of a double stranded portion of a DNA molecule, whereas others catalyze the cleavage of both strands, to catalyze the winding and/or unwinding of DNA. Topoisomerases may be sequence specific, cleaving at or after a particular sequence, or non-sequence specific, not cleaving at a preferred sequence. Two sequence specific topoisomerases are known, vaccinia virus DNA topoisomerase I and MCV topoisomerase. Both cleave one DNA strand immediately after the sequence CCCTT (SEQ ID NO: 1), referred to herein as a “topoisomerase cleavage recognition site.”

As used herein, a “topoisomerase-nucleic acid bound intermediate” is generated by providing a double stranded DNA substrate with a topoisomerase cleavage recognition site close to the 3′ end of one of the strands of the double stranded portion of the DNA. Cleavage of the strand results in the production of a short cleavage product that is too short to continue to be stably hybridized to the other DNA strand (Tₘ is no more than 10° C. higher, preferably no more than 5° C. higher than the temperature of the topoisomerase reaction). The topoisomerase remains bound to the nucleic acid until a nucleic acid to complete the substrate for ligase activity anneals to the compatible cohesive end to allow for ligation and release of the topoisomerase.

Ranges provided herein are understood to be shorthand for all of the values within the range. For example, a sequence of 1 to 50 nucleotides in length is understood to include nucleotide sequences of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

Unless specifically stated or obvious from context, as used herein, the term “or” is understood to be inclusive.

Unless specifically stated or obvious from context, as used herein, the terms “a”, an and “the” are understood to be singular or plural.

### BRIEF DESCRIPTION OF THE DRAWINGS

FIG. 1 is schematic of the method of the invention using a topoisomerase as the enzyme.

FIG. 2 is a schematic of the method of the invention using a ligase as the enzyme.

FIG. 3 is a schematic of the method of the invention using a reverse transcriptase as the enzyme.

FIG. 4 is a schematic of the topoisomerase ligation assay.

FIG. 5 is an amplification plot demonstrating the sensitivity of the detection of the topoisomerase ligation assay.

FIG. 6 is a graph of the threshold cycle number (Cₜ) with varying concentrations of topoisomerase.

FIG. 7 is a standard curve showing the Cₜ over seven orders of magnitude of topoisomerase concentration.

FIG. 8 is a standard curve of Cₜ over six orders of magnitude of topoisomerase concentrations demonstrating that fusion of protein-G to vaccinia virus DNA topoisomerase I does not substantially interfere with the function of the enzyme and does not disrupt the linearity of the result over six orders of magnitude.

FIGS. 9A and 9B are graphs of Ct of varying amounts of VEGF as determined by ELISA using topoisomerase as a reporter enzyme.

FIG. 10 is a graph of Cₜ with varying concentrations of His-tagged T3 ligase in the presence or absence of ATP.

FIG. 11 is a graph of Cₜ with varying concentrations of His-tagged T3 ligase in the presence or absence of ATP with the reaction carried out in PCR mastermix.

### DETAILED DESCRIPTION OF THE INVENTION

The present invention provides methods, kits and compositions for the detection of an analyte. In the methods of the invention, the presence of an analyte in a reaction results in formation of a nucleic acid amplification template indicative of the presence of analyte in the reaction. The enzyme of the analyte-specific binding agent interacts with one or more polynucleotide molecules to produce an amplification template molecule, preferably a PCR amplification template. Amplification and detection of the amplification product is a sensitive and potentially quantitative indicator of the presence of the analyte.

In a first aspect of the invention, the invention provides a method for detecting an analyte in a sample by contacting the analyte with an analyte-specific detection agent that specifically binds the analyte in the sample, and further includes an enzymatic portion that is capable of catalyzing the formation of a template for PCR amplification. The template for PCR amplification can be generated by the ligation of at least one strand of an annealed pair of DNA duplexes with cohesive compatible ends to form a linear DNA polynucleotide. More than one pair of DNA duplexes can be ligated in tandem to create a chain of three or more ligated duplexes. The cohesive, compatible ends can be generated at least, in part, by a sequence specific topoisomerase that cleaves one of the strands, releasing the cleavage product and generating a cohesive end. The template for PCR can also be generated by a reverse transcriptase in the presence of an RNA template. The cDNA produced is a template for PCR. The template for PCR is contacted with a first and a second primer in an amplification reaction. The first primer binds specifically to the template for PCR amplification under conditions to allow for amplification to generate a complementary strand to the template. This second strand binds the second primer for amplification of the second strand by PCR. The primers are designed to allow the amplification of a specific product from the template to demonstrate the presence of the analyte in the sample. The generation of the amplification product can be monitored by the use of, for example, SYBR Green, TaqMan® probes, or Sentinel® Molecular Beacons probes. The amount of signal detected in the unknown samples can be compared to the signal detected in the control samples containing a known amount of analyte to determine the amount of analyte present in the original unknown sample. Alternatively, the amplification product may be detected semi-quantitatively or qualitatively, for example, by dot blot or gel electrophoresis and ethidium bromide staining. By analyzing a series of dilutions of the unknown sample and comparing it to a series of known samples, the amount of analyte sample in the unknown sample can be estimated.

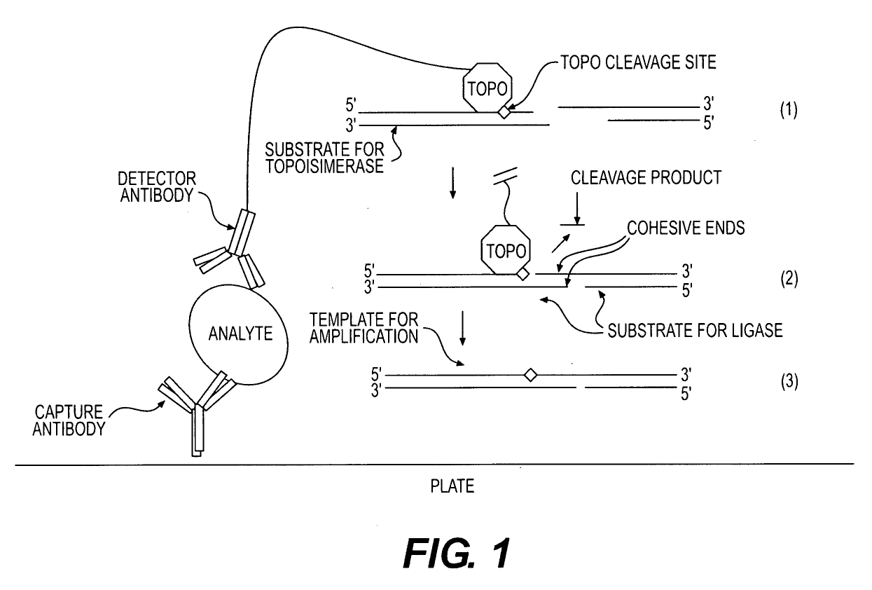

Schematics of various embodiments of the methods of the invention are provided in FIGS. 1, 2, and 3 using a topoisomerase, a ligase, and a reverse transcriptase as the enzyme, respectively.

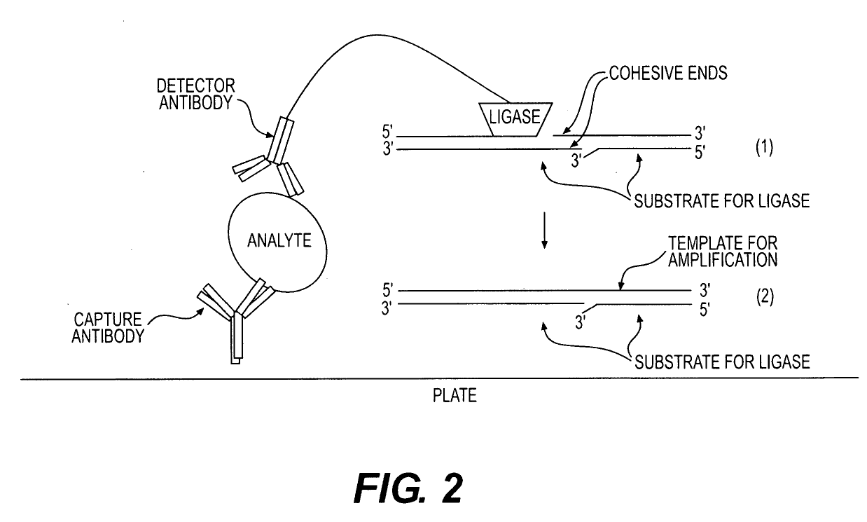

In FIG. 1 (1), the topoisomerase portion of the bound analyte-specific binding agent is bound to the topoisomerase specific cleavage site on the substrate for topoisomerase. For clarity, the complete bound analyte specific binding agent is shown only once; however, the topoisomerase remains attached to the binding agent throughout the method. In FIG. 1 (2), the topoisomerase cleaves the topoisomerase substrate to generate compatible cohesive ends to generate the ligase substrates. A cleavage product is released generating a topoisomerase-nucleic acid bound intermediate. The cohesive ends of the ligase substrates anneal, allowing for ligation of the top strand of each of the annealed substrates and release of the topoisomerase. The 3′ end of the bottom strand need not be fully complementary to the top strand (see, e.g., FIG. 2 (1)). FIG. 1 (3) shows the ligation product that can serve as a template in an amplification reaction.

In FIG. 2 (1), the ligase portion of the bound analyte-specific binding agent is bound to one half of a substrate for ligase, and the cohesive ends of the ligase substrates are annealed. The 5′ end of at least one of the cohesive ends is phosphorylated. In FIG. 2 (2) the ligase has joined the strands having the 5′ phosphorylated end to generate a template for use in an amplification reaction.

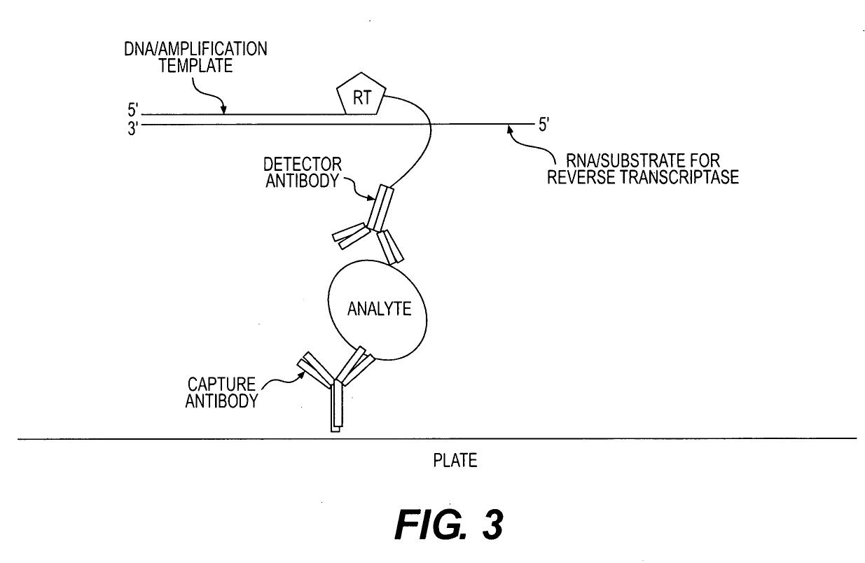

In FIG. 3, the reverse transcriptase portion of the bound analyte-specific binding agent is bound to RNA which is the substrate for reverse transcriptase. The reverse transcriptase transcribes the RNA strand into DNA to generate a template for use in an amplification reaction.

The invention features methods useful for the specific and sensitive detection and optionally quantitation of an analyte in a sample. The methods of the invention can be used to detect essentially any analyte provided that a specific analyte can be prepared to bind the analyte of interest. The analyte can be derived from a biological sample such as a tissue or bodily fluid from an organism such as a mammal or human. The methods can be used, for example, to monitor expression of protein over time to determine disease status or the efficacy of a clinical intervention. The sample can be from an environmental source, for example to detect the presence of an analyte in a water or soil sample. The sample can be from an agricultural or food source to test for the presence of contaminants or infectious agents. Methods of preparing extracts for binding of analytes to specific analyte-binding moieties are well known to those skilled in the art.

In an aspect, the invention features compositions and kits for use as detection agents in combination with known ELISA type assays. The analyte specific binding agents can be used in combination with substrates for the generation of a template for an amplification reaction, and reagents required for use in an amplification reaction, preferably a PCR reaction, more preferably a quantitative PCR reaction.

A number of kits for detection of specific analytes are commercially available. Such kits typically include an ELISA plate and a capture molecule, either pre-coated on the plate or separately, to bind the analyte. Control analyte at a known concentration can be provided with the kit as a positive control. An analyte-specific antibody for detection of the analyte is provided as a component of the commercial ELISA, and a second antibody bound to a detectable label is provided to detect the analyte-specific antibody. In an embodiment, the kits of the invention include compositions and reagents for use in lieu of the second antibody provided and/or typically use in the ELISA assays.

The invention provides kits containing an enzyme coupled to an antibody binding domain such as Protein A, G, or L for use as a detection reagent with ELISA assays, including commercially available kits. The enzyme can also be attached to streptavidin or avidin for attachment to a biotinylated antibody included in the kit or obtained from another source. The enzyme coupled to the antibody binding domain or streptavidin can be contacted with the analyte specific antibody or any biotinylated molecule, respectively, to generate an analyte-specific detection agent. Alternatively, the enzyme coupled to the antibody binding domain can be contacted with an antibody that binds the analyte specific antibody. For example, if the analyte specific antibody is a mouse IgG monoclonal antibody, a commercially available anti-mouse IgG antibody can be contacted with the enzyme coupled to the antibody binding domain (e.g., Protein G). Alternatively, kits including anti-immunoglobulin antibodies coupled to enzymatic moieties for use as detection agents can be included in kits of the invention.

The kits further include nucleic acid molecule(s) that are a substrate for the enzyme to generate a template for PCR.

In a topoisomerase based kit, the nucleic acid molecules can be one or two double stranded nucleic acid molecules, preferably DNA molecules, one end of which contains a topoisomerase cleavage site which, when cleaved, produces a compatible cohesive end for annealing to the second double stranded nucleic acid molecule. The acceptor strand includes a 5′-OH to form a substrate for the topoisomerase. The strand that does not include the specific topoisomerase cleavage site can include modifications to the sugar, base or backbone to prevent amplification of the strand by a polymerase with a high level of discrimination, such as Pfu. Modifications that block amplification by Pfu include, but are not limited to, methoxy sugar modifications, or non-standard DNA bases such as uracil. Modifications are incorporated into the 3′ end of the strand that is not a substrate for the ligase.

In a ligase based kit, the nucleic acid molecules can be two double stranded nucleic acid molecules, preferably DNA molecules, having compatible cohesive ends for annealing to the second double stranded nucleic acid molecule. At least the acceptor strand in the strand to be a template for amplification includes a 5′-phosphate to form a substrate for the topoisomerase. The strand that does not include the 5′-phosphate can include non-natural dNTPs to prevent amplification of the strand by a polymerase with a high level of discrimination, such as Pfu.

In a reverse transcriptase based kit, the nucleic acid molecule is an RNA molecule. The RNA molecule can include chemical modifications to increase the stability of the RNA template without substantially interfering with the reverse transcriptase. Such modifications are known to those skilled in the art.

The kits can further provide at least one of primers, probes (e.g., TaqMan® or Sentinel® Molecular Beacon probes), or other agents (e.g., SYBR Green) for the quantitative amplification of a product from the template generated by the enzymatic moiety.

In an aspect, the invention further provides compositions for use in the methods and kits of the invention. For example, the invention provides analyte-specific detection agents having an analyte-specific binding moiety coupled to an enzymatic moiety. The invention further provides an enzymatic moiety coupled to the antibody binding domain such as Protein A, G, or L, preferably for use as a detection agent. The invention also provides an enzymatic moiety coupled to the antibody binding domain further coupled to an anti-immunoglobulin antibody, preferably for use as a detection agent.

In an aspect of the invention, the method includes preparing a solid surface with an analyte capture molecule to allow for binding of a specific analyte that may be present in the sample. Commercially available antibody coated plates can be used, or ELISA kits including antibodies and instructions for binding of the antibody to the surface can also be used. The analyte capture molecule can be a specific or non-specific binding agent, or the specific agent can be bound to the plate by a non-specific agent. The capture molecule can include one half of a binding pair, such as biotin-avidin or biotin-strepavidin. The solid surface can be coated with avidin or streptavidin, and the capture molecule can be linked to biotin. The exact method of attaching the capture molecule to the solid support is not a limitation of the invention. After coating the plate with the capture molecule, the surface is washed and blocked with a non-specific agent to prevent non-specific binding of the agent to the surface.

The prepared plate is contacted with the analyte in the appropriate buffer under the appropriate conditions of time and temperature to allow for binding. These conditions may vary depending on the analyte and capture reagent used. Such considerations are well understood by those skilled in the art. Unbound analyte is removed by washing.

An analyte-specific binding agent is prepared for detecting the presence of the analyte. The analyte-specific binding agent includes an analyte specific binding molecule coupled, fused, or otherwise attached to an enzyme. The analyte-binding molecule must be selected such that it binds the analyte at an epitope distinct from the analyte capture molecule, and it does not bind to any other component of the reaction other than the analyte and the enzymatic moiety. For example, the capture molecule and the analyte binding moiety can be monoclonal antibodies targeted to the analyte that bind at two discrete, non-interfering epitopes on the analyte. Alternatively, the capture molecule and the analyte binding molecule can both be polyclonal antibodies directed to at least a substantial portion of the analyte such that two antibodies can bind to the analyte simultaneously. Antibodies that bind to the same epitope can be used for analytes that have repeating structures or motifs (e.g., collagen).

The enzyme portion of the analyte specific binding agent can be a ligase derived from either a ligase or a topoisomerase, or a reverse transcriptase moiety. As the analyte-specific binding molecule is frequently an antibody, the analyte-specific binding molecule can often be conveniently coupled to the enzyme by expressing the enzymatic moiety as a fusion protein with a generic antibody binding peptide such as protein A, G, or L. However, care must be taken to insure that the enzyme does not bind to the capture molecule attached to the solid support. For example, chicken IgY is not bound by any of Protein A, G, or L, and total IgG from rat, cow, goat, and sheep are only weakly bound by Protein A, whereas human, mouse, and rabbit IgG are strongly bound by Protein A. Therefore, a chicken IgY antibody or a rat, cow, goat, or sheep antibody can be used as an antibody capture molecule in conjunction with an enzyme fused to a Protein A domain for binding to a human, mouse, or rabbit IgG as an analyte-specific binding moiety. Such allowable combinations can be readily determined by those skilled in the art.

The analyte-specific binding agent is contacted with the analyte bound to the prepared solid surface under conditions to permit binding of the analyte to the agent. These conditions may vary depending on the analyte and the binding agent used. Such considerations are well understood by those skilled in the art. Unbound binding agent is removed by washing.

The subsequent steps and reagents are dependent upon the enzyme moiety included in the enzyme. Appropriate substrates and reaction conditions for each topoisomerase, ligase, and reverse transcriptase are discussed herein. The bound analyte-specific binding agent is incubated with the appropriate nucleic acid substrate under conditions to allow the reaction catalyzed by the enzyme to take place. After incubation, a portion of the reaction mixture is transferred to an amplification reaction mixture, preferably a PCR reaction mixture, more preferably a quantitative PCR reaction mixture. The amount of amplification product is detected during and/or after the amplification reaction, and the presence of the analyte in the sample is determined.

Binding Molecules

The methods of the present invention can be adapted for the detection of any analyte by simply altering the capture molecule (e.g., the capture antibody attached to the solid support) and/or the analyte-specific detection agent used in the method such that the capture and detector molecules utilized specifically recognize and bind the analyte for which the method is being used. In some embodiments, the analyte may be directly bound to the solid support so that a capture antibody is not necessary. In other embodiments, a capture antibody binds the analyte, an unlabeled intermediate antibody binds the analyte, and a detector antibody binds the intermediate antibody.

In other embodiments, the assay is performed as a solution-phase reaction (e.g., without a capture antibody and solid-support, see FIGS. 1B, 2B, and 3B). In these embodiments, the method generally utilizes two analyte specific binding molecules (e.g., antibodies), the first operatively coupled to a polynucleotide template and the second coupled to the enzymatic moiety. The antibodies are designed so as to bind within close proximity to one another on the analyte so as to allow the polynucleotide and the enzymatic moiety to interact so as to form an amplification template. Such assays are described in U.S. application Ser. No. 11/546,695, filed Oct. 11, 2006 and herein incorporated by reference in its entirety.

The capture molecule and the analyte-specific detection agent may recognize and bind the same portion or epitope of the analyte under investigation (e.g., multivalent analyte). Alternatively, the capture molecule and the analyte-specific detection agent recognize and bind different portions or epitopes of the analyte. In some embodiments, the capture molecule and analyte-specific detection agent may not bind to the same analyte but two different analytes that interact to form a complex. For example, a capture antibody may be specific for and bind to a receptor protein and the detector antibody may be specific for and bind to a ligand of the receptor such that the capture molecule, receptor protein, ligand and detector antibody all form a complex.

The specific molecules used as the capture molecule and the detector molecules used in the methods of the present invention are not particularly limited. Molecules useful as the capture and analyte-binding detector molecules include monoclonal, polyclonal, or phage derived antibodies, antibody fragments, peptides, ligands, haptens, nucleic acids, nucleic acid aptamers, protein A, protein G, folate, folate binding proteins, plasminogen, maleimide and other sulfhydryl reactive groups, and those that may be produced for use with the methods of the present invention.

Preferably, the capture and analyte-binding detector molecules are monoclonal, polyclonal, or phage derived antibodies, or antibody fragments. More preferably, the capture and detector molecules are monoclonal antibodies.

Antibodies, whether they are polyclonal, a monoclonal or an immunoreactive fragment thereof, can be produced by customary methods familiar to those skilled in the art. Conventional monoclonal and polyclonal antibodies are of use and represent a preferred type binding molecule. Established methods of antibody preparation therefore can be employed for preparation of the immune type binding molecules. Suitable methods of antibody preparation and purification for the immune type binding moieties are described in Harlow and Lane in Antibodies a Laboratory Manual, Cold Spring Harbor Laboratory Press, Cold Spring Harbor, N.Y. (1988). Furthermore, the assays described herein can be used with currently available commercially available antibodies.

“Polyclonal antibodies” are heterogeneous populations of antibody molecules derived from the sera of animals immunized with an antigen, or an antigenic functional derivative thereof. For the production of polyclonal antibodies, host animals such as rabbits, mice and goats, may be immunized by injection with an antigen or hapten-carrier conjugate optionally supplemented with adjuvants.

Any method known in the art for generating monoclonal antibodies are contemplated, for example by in vitro generation with phage display technology and in vivo generation by immunizing animals, such as mice, can be used in the present invention. These methods include the immunological methods described by Kohler and Milstein (Nature 256, 495-497 (1975)) and Campbell (“Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam (1995)); as well as by the recombinant DNA method described by Huse et al. (Science 246, 1275-1281 (1989)). Standard recombinant DNA techniques are described in Sambrook et al. (Molecular Cloning, Second Edition, Cold Spring Harbor Laboratory Press (1987)) and Ausubel (Current Protocols in Molecular Biology, Green Publishing Associates/Wiley-Interscience, New York (1990)). Each of these methods is incorporated herein by reference.

The capture molecule and the detector molecule are not limited to intact antibodies, but encompass other binding molecules such as antibody fragments and recombinant fusion proteins comprising an antibody fragment.

### Coupling Analyte-Specific Binding Molecules and Enzymes

The analyte-specific binding molecule and the enzyme can be attached or coupled in any way as long as the attachment or coupling does not substantially interfere with the activity of either of the moieties. The exact method or structure providing the coupling between the two portions is not a limitation of the invention and is a matter of choice depending on the various moieties selected and the reagents available to the end user. One coupling type comprises an enzyme coupled to an analyte specific binding molecule. These may be prepared using methods well known to those skilled in the art. D. G. Williams, J. Immun. Methods, 79, 261 (1984). Alternatively, analyte-binding agents can be generated using recombinant DNA and genetic engineering techniques. I. Pastan and D. Fitzgerald, Science, 254, 1173 (1991).

Extensive guidance can be found in the literature for covalently linking proteins to binding compounds (other proteins), such as antibodies, e.g. Hermanson, Bioconjugate Techniques, (Academic Press, New York, 1996), and the like. In one aspect of the invention, one or more enzymes are attached directly or indirectly to common reactive groups on an analyte-specific binding molecule. Common reactive groups include amine, thiol, carboxylate, hydroxyl, aldehyde, ketone, and the like, and may be coupled to proteins by commercially available cross linking agents, e.g. Hermanson (cited above); Haugland, Handbook of Fluorescent Probes and Research Products, Ninth Edition (Molecular Probes, Eugene, Oreg., 2002). In one embodiment, an NHS-ester of a molecular tag is reacted with a free amine on the binding molecule.

Another type of coupling consists of a polynucleotide template sequence coupled to an enzyme when the analyte is a nucleic acid. These can be prepared using variations of methods known to those skilled in the art for linking proteins to amino-oligonucleotides. For example, this may be accomplished using enzymatic tailing methods in which an amino-modified dNTP is added onto the 3′ end of the nucleic acid. A. Kumar, Anal. Biochem., 169, 376 (1988). Alternatively, amino-modified bases can be synthetically introduced into the nucleic acid base sequence. P. Li, et al., Nucleic Acids Res., 15, 5275 (1987). Enzymes can then be attached to amino-modified nucleic acids in the method of Urdea (M. S, Urdea, Nucleic Acids Res., 16, 4937 (1988).

In some embodiments, the nucleic acid/antibody conjugates involves the coupling of heterobifunctional cross-linkers to the DNA oligonucleotide targets which in turn are coupled to antibodies using chemistry described by Tseng et. al. in U.S. Pat. No. 5,324,650.

To facilitate the chemical attachment of the oligonucleotides to the antibodies, the oligonucleotides may be amino-modified by introducing a primary amine group at their 5′ end during synthesis using cyanoethyl-phosphoramidite chemistry. The amino-modified oligonucleotides may be further modified with a hetero-bifunctional reagent that introduces sulfhydryl groups. The reagent, N-succinimidyl S-acetylthioacetate (SATA) is a heterobifunctional cross-linker agent that uses the primary amine reactive group, N-hydroxyl-succinimide (NHS) to couple to the amino-modified oligonucleotides introducing an acetyl-protected sulfhydryl group. The antibodies are modified with another NHS cross-linking agent, succinimidyl 4-(N-maleimidomethyl)cyclohexane-1-carboxylate (SMCC). The SMCC reacts with primary amine groups within the peptides (e.g., the epsilon-groups on lysine) of the antibody, introducing a maleimide group (a free sulfhydryl reactive group) to the antibody. The maleimide-modified antibodies are mixed with the SATA modified antibodies. The acetyl-protected sulfhydryl groups on the SATA-modified oligonucleotides are activated with the addition of hydroxylamine to produce reactive, free sulfhydryl groups (U.S. Pat. No. 5,324,650). The free sulfhydryl-containing oligonucleotides react immediately with maleimide-modified antibodies forming DNA to antibody conjugates.

Alternatively, the enzyme is attached to an antibody analyte-specific binding portion of an antibody analyte specific binding agent by a protein A, protein G, or protein L coding sequence fused to the enzyme sequence (see, e.g., examples below), or expressed as two separate polypeptides and chemically joined.

Similarly, a streptavidin or avidin sequence can be linked to the enzyme by fusion of the coding sequence to the coding sequence of the enzyme, or the polypeptides can be expressed separately and chemically joined. The streptavidin or avidin linked enzyme can then be mixed with any biotinylated molecule, such as a polypeptide or nucleic acid molecule under conditions well known to those of skill in the art.

### Binding Molecules Attached to Solid Surface

As used herein, a “solid support” or “solid surface” refers to any structure that provides a support for the capture molecule. Suitable solid supports include polystyrene, derivatized polystyrene, a membrane, such as nitrocellulose, PVDF or nylon, a latex bead, a glass bead, a silica bead, paramagnetic or latex microsphere, or microtiter well. As a further example, the solid support may be a modified microtiter plate, such as a Top Yields plate, which allows for covalent attachment of a capture molecule, such as an antibody, to the plate. When the solid support is a material such as a bead, paramagnetic microsphere or latex microsphere, the solid support may be contained in an open container, such as a multi-well tissue culture dish, or in a sealed container, such as a snap-top or screw-top tube, all of which are commonly used in laboratories.

In one embodiment, a capture antibody is bound to a solid support. In an alternative embodiment, the analyte binds directly to the solid support.

The solid support may be modified to facilitate binding of the capture molecule to the surface of the support, such as by coating the surface with poly L-lysine, or siliconized with amino aldehyde silane or epoxysilane. The skilled artisan will understand that the circumstances under which the methods of the current invention are performed will govern which solid supports are most preferred and whether a container is used. Commercial, precoated ELISAs plates and ELISA kits are commercially available and can be used in conjunction with the detection methods of the instant invention.

Quantities of the capture molecule to be attached to the solid support may be determined empirically by checkerboard titration with different quantities of analyte that would be expected to mimic quantities in a test sample. Generally, the quantity of the analyte in the test sample is expected to be in the attogram to milligram range. An unknown concentration of the analyte in a test sample will be added at specified volumes, and this will influence the sensitivity of the test. If large volumes of the test sample (e.g., 200-400 uL) are used, modification of the test format may be needed to allow for the larger sample volumes. Generally, however, the concentration of the capture molecule will be about 1 to about 10 micrograms per mL.

The capture molecule can be attached to a solid support by routine methods that have been described for attachment of an analyte to plastic or other solid support systems (e.g., membranes or microspheres). Examples of such methods may be found in U.S. Pat. No. 4,045,384 and U.S. Pat. No. 4,046,723, both of which are incorporated herein by reference.

Attachment of the capture molecule to surfaces such as membranes, microspheres, or microtiter wells may be performed by direct addition in PBS, or other buffers of defined pH, followed by drying in a convection oven.

The capture molecule may be attached to the solid support by an attachment means, such as via adsorption, covalent linkage, avidin-biotin linkage, streptavidin-biotin linkage, heterobifunctional cross-linker, Protein A linkage or Protein G linkage. Each of the attachment means should permit the use of stringent washing conditions with minimal loss of the capture molecule from the surface of the solid support. Such conditions are discussed below and well understood by those of skill in the art. As an example, the adsorption may be hydrophilic adsorption. As a further example, the heterobifunctional cross-linker may be maleic anhydride, 3-aminopropyl trimethoxysilane (APS), N-5 azido, 2-nitrobenzoyaloxysuccinimide (ANB-NOS) or mercaptosilane.

The capture molecule may be attached to the solid support though a portion of the capture molecule, such as an amino acid residue, preferably a lysine or arginine residue, a thiol group or a carbohydrate residue. When the capture molecule is an antibody, the thiol group may be a thiol group of the antibody hinge region.

The solid support may be derivatized with avidin or streptavidin, and the capture molecule may be modified to contain at least one biotin moiety, to aid in the attachment of the capture molecule to the solid support. Alternatively, the solid support may be derivatized with biotin, and the capture molecule may be modified to contain at least one avidin or at least one streptavidin moiety.

### Test Sample and Analyte Binding

In practicing the methods of the present invention, a sample suspected of containing the selected analyte under investigation is applied to a prepared support coated with an antibody or other agent to capture the analyte on the solid surface. Alternatively, in a solution based assay the test sample is directly added to the solution phase reaction mixture that does not include a solid support. Depending on the identity of the support, the support may be contained within a culture device of some type. When the support is a membrane, for example, a shallow glass dish slightly bigger that the length and width of the membrane may be used. When the support is a microsphere, the microspheres may be contained in a tube, such as a polypropylene or polystyrene screw-top tube. The identity of the container is not critical, but it should be constructed of a material to which the reagents used in the methods of the present invention do not adhere non-specifically.

The quantity of test sample used is not critical, but should be an amount that can be easily handled and that has a concentration of analyte that is detectable within the limits of the methods of the present invention. The test sample should also be sufficient to adequately cover the support, and may be diluted if needed in this regard. For example, the quantity of the test sample may be between 0.5 uL and 2 mL. Preferably, the quantity of the test sample is between 0.5 uL and 1 mL. Most preferably, the quantity of the test sample may be between 0.5 uL and 200 uL. Smaller volumes of sample can be used in conjunction with microfluidics devices.

While the concentration of the analyte in the test sample is not critical, it should be within the detection limits of the methods of the present invention. The skilled artisan will understand that the concentration may vary depending on the volume of the test sample, and thus it is difficult to provide a concentration range over which an analyte may be detected.

The methods and kits taught herein can thus be used to detect analyte present in a sample at low numbers. A test sample analyzed in the method of the invention can contain 10⁴ molecules of the analyte or less, 10⁶ molecules of the analyte or less, 10⁸ molecules of the analyte or less, 10¹⁰ molecules of the analyte or less, 10¹² molecules of the analyte or less, 10¹⁴ molecules of the analyte or less, 10¹⁶ molecules of the analyte or less, 10¹⁸ molecules of the analyte or less, 10²⁰ molecules of the analyte or less, or 10²² molecules of the analyte or less.

The capture molecule is incubated with the solid support for a period of time sufficient to allow the capture molecule to bind the solid support. Alternatively an analyte is incubated with the solid support for a period of time sufficient to allow the analyte to bind the solid support. Preferably, the incubation proceeds from between about 10 minutes and about 60 minutes, but may require overnight.

The particular temperature at which each of the incubation steps of the methods is performed is also not critical. The temperature depends, for example, on the enzyme used at any particular step or the Tₘ of the nucleic acids to be annealed at any particular step. Such considerations are well understood by those skilled in the art.

### Detecting Bound Analyte-Specific Binding Agent

The detection reaction may be performed in the same or a separate reaction vessel as the binding/ligation or reverse transcriptase reaction. For example, an aliquot of the reaction mixture having the amplification template is transferred to a corresponding well of a 96-well PCR plate. In this step, the amplified template is reacted with a detection reagent (e.g., TaqMan® probe, SYBR Green dye), a first and second primer and a polymerase. The detection reaction mixture is subjected to reaction conditions that allow the annealing of the primers, amplification of the amplified template and detection of the amplified template. In a preferred embodiment, the first and second oligonucleotide primers are different. In an alternative embodiment, the first and second oligonucleotide primers are the same. For example, in one embodiment the detection reaction is run in a real-time PCR device that is programmed with the appropriate times and temperatures necessary for amplification and detection. For example a MX3005P real-time PCR device may be utilized with the program corresponding to a SYBR Green detection assay with dissociation curve and a 2-step cycling parameter of 95° C. for 10 minutes, followed by 40 cycles of 95° C. for 15 seconds, and 63° C. for 45 seconds. Other means of real-time PCR detection are well known in the art (e.g., TaqMan® and Sentinel™ Molecular Beacon detection assays) and can be adapted for use in the present embodiment. The detected signal can then be used to determine the concentration of the analyte in the sample.

### Preventing Amplification of Non-Specific Amplification Products

In the ligation based methods of the invention, only one strand needs to be ligated to generate an amplification product for PCR. Using topoisomerase, only one strand is ligated. However, when at least a portion of the ligation or topoisomerase reaction mixture is transferred to the amplification reaction mixture, it is possible that the unligated portions of the double stranded DNA molecules added to the ligation or topoisomerase reaction could result in the production of non-specific amplification products. Formation of such products can be limited by incorporation unpaired 3′ nucleotide overhangs or incorporation of modifications into the strand such as non-natural nucleotide analogs, such as 2′-Me or similar modifications, UTP or other RNA nucleotides, specifically the 3′ end of the non-ligated strand proximal to the ligation site, coupled with the use of a polymerase, such as Pfu, that has a high level of discrimination and will not incorporate nucleotides across from modified DNA strand.

### Wash Conditions

Between the addition of reagents in the methods of the present invention, the assay system is preferably subjected to washing to reduce the incidence of non-specific binding. While the number of wash cycles and soak times is empirically determined, in general either water or a low or high molarity salt solution (up to about 1 M salt, typically NaCl) with a detergent, typically a non-ionic polymeric detergent such as Tween 20, Triton X-100, or NP-40 (up to about 1.0%) may be used as the washing solution. 1-8 washes, each lasting about 5 seconds to 5 minutes may be performed, after incubation of each of the reagents used in the methods. It is understood that very high or very low salt concentrations are more stringent than physiologic salt concentrations. Higher detergent concentrations are more stringent than lower detergent concentrations. Selection of an appropriate wash buffer is well within the ability of those skilled in the art. Some commonly used wash buffers include phosphate buffered saline (PBS) (137 mM NaCl, 2.7 mM KCl, 10 mM Na₂HPO₄, 2 mM KH₂PO₄) or Tris buffered saline (TBS) (100 mM Tris-Cl, pH 7.5; 150 mM NaCl) with 0.1% Tween 20 or 0.1% Triton X-100. Washing can be performed between each incubation step, e.g., after addition of the capture molecule to the solid support, after addition of the test sample and after addition of the detector molecule. Exemplary washing conditions are described in the Examples.

### Diagnostics

The methods, kits, and compositions of the invention can be used for the detection and/or quantification of an analyte in a sample. Typical analytes may include, but are not limited to proteins, peptides, cell surface receptors, receptor ligands, nucleic acids, carbohydrates, haptens, molecules, cells, microorganisms and fragments thereof. Due to the sensitivity of the methods of the invention, the methods can be used for the detection of therapeutic biological agents that are typically present at a very low concentration.

### Recombinant Polypeptide Expression

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example Substrates for Topoisomerase/Ligase

For topoisomerase, the first double stranded nucleic acid comprises the sequences below. For ligase, the top strand does not include the ATGGGT sequence at the 3′ end.

(SEQ ID NO: 2)

5′-TGACGCCCGAAGCCAAGTGCGGGACGGCTTCTCCAGCTTGGCCCCTT
ATGGGT-3′

(SEQ ID NO: 3)

3′-ACTGCGGGCTTCGGTTCACGCCCTGCCGAAGAGGTCGAACCGGGGAA
TACCCTTGCT-5′

and the second double nucleic acid molecule comprises:

(SEQ ID NO: 4)

5′-ATGGGAACGAGCAGACCGACCGCTAGACAGCTCCGTGGA-3′

(SEQ ID NO: 5)

3′-CGTCTGGCTGGCGATCTGTCGAGGCACCT-5′.

First and second oligonucleotide primers for amplification that can be used with the pair of substrates for topoisomerase/ligase shown above include a first oligonucleotide primer 5′-TCCACGGAGCTGTCTAGCG-3′ (SEQ ID NO: 10) and a second oligonucleotide primer 5′-TGACGCCCGAAGCCAAGTG-3′ (SEQ ID NO: 11).

### Example Substrate for Reverse Transcriptase

Sequence is provided for an RNA strand to be a substrate for reverse transcriptase. The sequence can be the product of a reverse transcription reaction with the DNA template being a reverse complement RNA strand shown.

(SEQ ID NO: 9)

5′GAUUGGAGCUCCACCGCGGUGGCGGCCGCUCUAGAACUAGUGGAUCCC
CCGGGCUGCAGGAAUCGAUAUCAAGCUAUCGAUACCGUCGACCUCGAGGG
GGGGCCGGUACCCCAGCUUUUGUUCCCUUUAGTGAGGGUUAAUUGCGCGC
UUGGCGUAAUCAUGGUCAUAGCUGUUUCCUGUGUGAAAU-3′.

### EXAMPLES

### Example 1

### Quantitative Detection of Topoisomerase Activity

The topoisomerase assay is based upon a two-step reaction of the vaccinia virus DNA topoisomerase I of MCV topoisomerase. The top strand of the substrate contains the topoisomerase recognition site (CCCTT). Topoisomerase reactions were performed by combining 1 μl topoisomerase and 2 μl substrate. Topoisomerase substrate and ligation substrate were each present a 10 nmoles/l in 1 mM Tris HCl, pH8.0, 60 mM NaCl, 1 ng/μl BSA. Reactions were allowed to incubate for 10 minutes at room temperature.

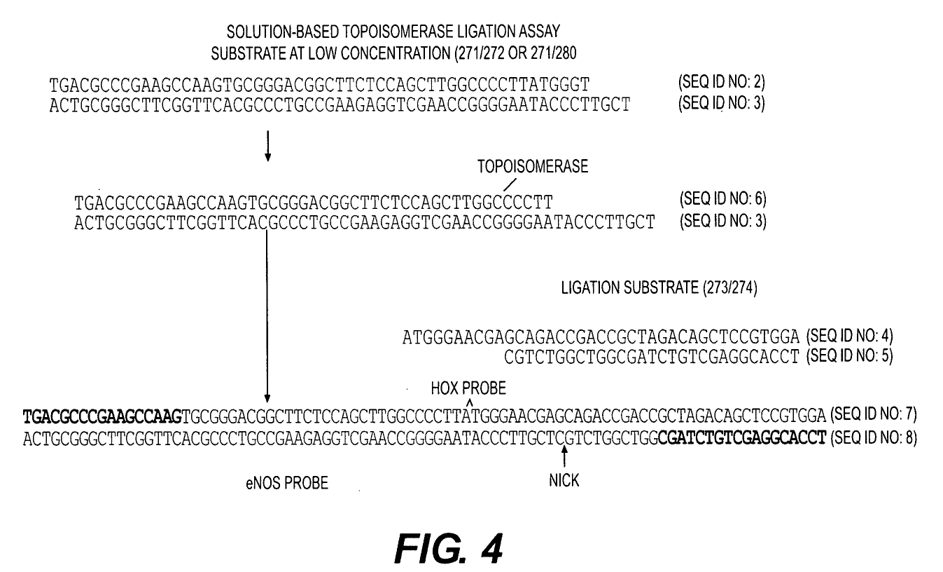

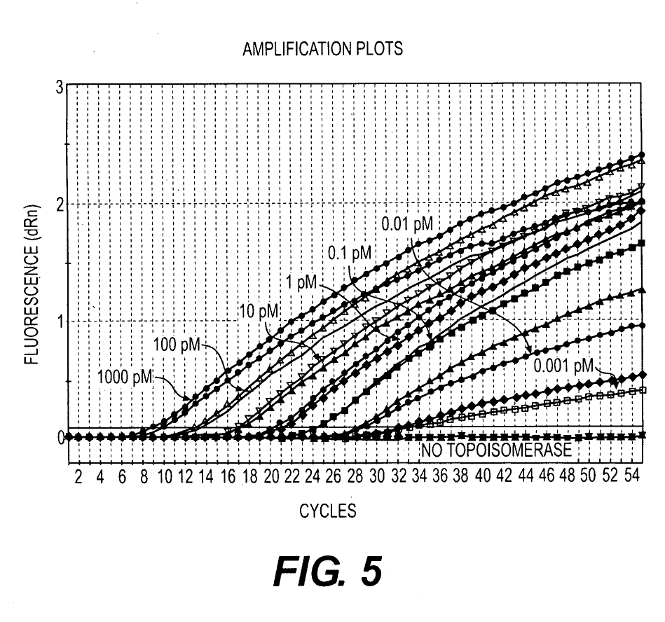

The topoisomerase enzyme cleaves the top strand after the recognition site and becomes covalently attached to the 3′-end of the cleavage site. The sequences 3′ to the cleavage site (In FIG. 4, the six-mer “ATGGGT” (SEQ ID NO: 12) of the 3′ end of the top strand) are too short to remain annealed to the complementing strand and the cleavage product will diffuse away from the substrate molecule. As a consequence, the topoisomerase reaction cycle (cleavage of one strand and subsequent ligation of the cleavage site) cannot be completed, and the topoisomerase enzyme becomes trapped as a topoisomerase-nucleic acid bound intermediate. The reaction cycle is completed by ligation to the ligation substrate, resulting in the product at the bottom of the flow chart. The product is then amplified with PCR primers that flank the ligated site within the newly-ligated DNA molecule (In FIG. 4, such sequences are indicated in bold. Note that the topoisomerase reaction only cleaves the top strand, leaving a nick at the bottom strand.)

For probe-based detection, two detection sequences were integrated in the product, to which probes had been established (hence the name Hox-probe and eNOS). It is noted that the Hox-probe sequence contains the topoisomerase recognition site (CCCTT). Quantitative PCR reactions were performed by addition of 27 μl qPCR mastermix (for probe-based detection: Brilliant® QPCR Master Mix, Cat. No. 600549, Stratagene; for dye-based detection: Brilliant® QPCR Core Reagent Kit, Cat. No. 600530, Stratagene) containing Taq polymerase and 400 nM of each PCR primer (final concentration). qPCR reaction cycles were performed for 10 minutes at 95° C., then 95° C. for 15 seconds and 60° C. for 45 seconds, for 40 cycles. For probe-based detection, the Taqman probe was added at 100 nM final concentration.

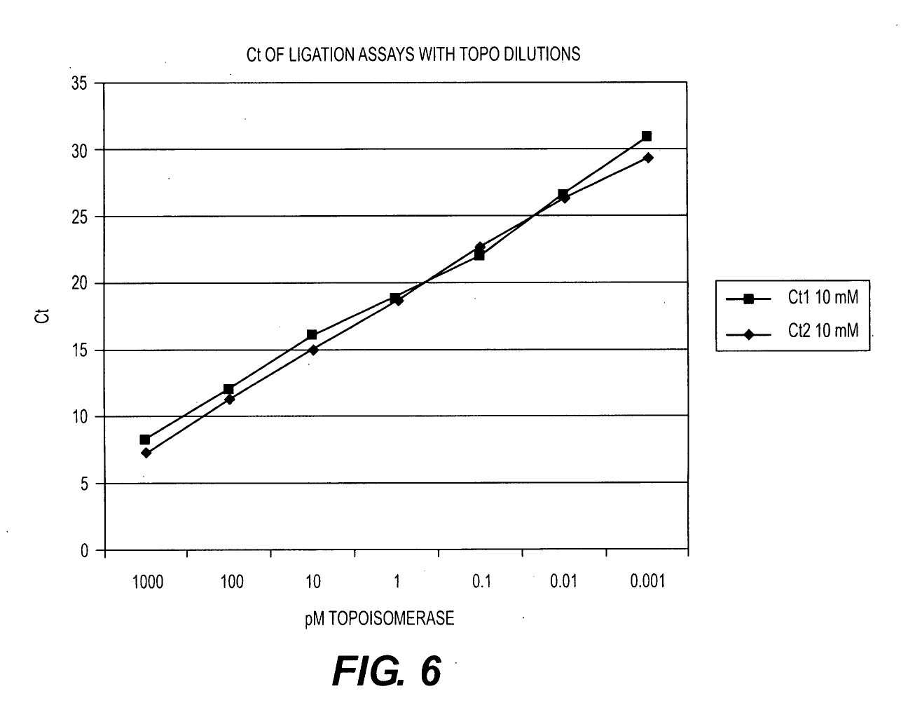

An amplification plot with Hox-probe based assay is shown in FIG. 6. A duplicate dilution series of vv-Topoisomerase in qPCR assay (note: no detectable Cₜ-value with no enzyme control). The data from Table 1 are shown graphically in FIG. 6.

TABLE 1

| pM Topo | Ct1 10 mM | Ct2 10 mM | Ct 50 mM |
|---|---|---|---|
| 1000 | 8.32 | 7.27 | 11.9 |
| 100 | 12.04 | 11.27 | 21.48 |
| 10 | 16.14 | 15.01 | 29 |
| 1 | 18.85 | 18.68 | 42.26 |
| 0.1 | 22.09 | 22.69 | No Ct |
| 0.01 | 26.64 | 26.34 | No Ct |
| 0.001 | 30.96 | 29.38 | No Ct |
| 0 | No Ct | No Ct | No Ct |

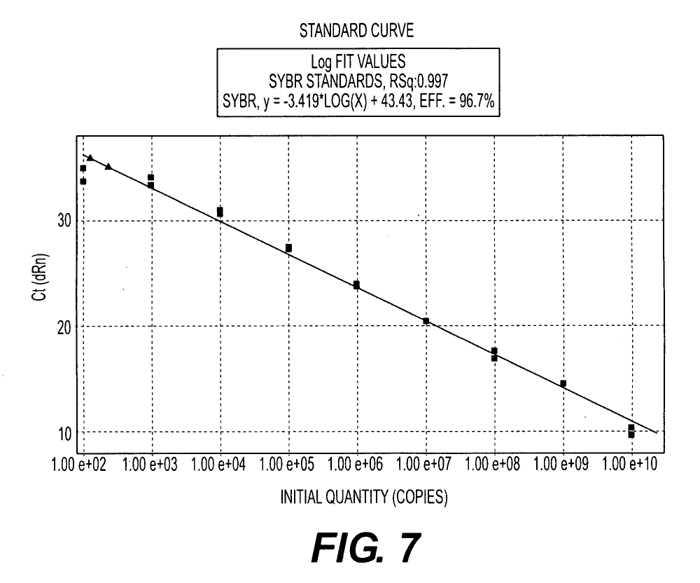

The same assay was performed and quantitated using SYBR Green rather than probe detection. The results were linear over four orders of magnitude. The data from Table 2 are shown graphically in FIG. 7.

TABLE 2

| Quantity (topoisomerase | |
|---|---|
| molecules) | Ct |
| 1.00E+10 | 8.3 |
| 1.00E+10 | 8.66 |
| 1.00E+09 | 13.46 |
| 1.00E+09 | 13.32 |
| 1.00E+08 | 15.91 |
| 1.00E+08 | 16.45 |
| 1.00E+07 | 19.36 |
| 1.00E+07 | 19.32 |
| 1.00E+06 | 22.97 |
| 1.00E+06 | 22.77 |
| 1.00E+05 | 26.36 |
| 1.00E+05 | 26.51 |
| 1.00E+04 | 29.99 |
| 1.00E+04 | 29.7 |
| 1.00E+03 | 33.23 |
| 1.00E+03 | 32.53 |
| 1.00E+02 | 34.12 |
| 1.00E+02 | 32.76 |
| 0.00E+00 | 35.01 |
| 0.00E+00 | 34.2 |
| no substrate | No Ct |
| no substrate | No Ct |

The detection limit of the assay was determined to be approximately 100 molecules of vv-Topoisomerase.

### Example 2

### Activity of Topoisomerase Protein-G Fusion Proteins

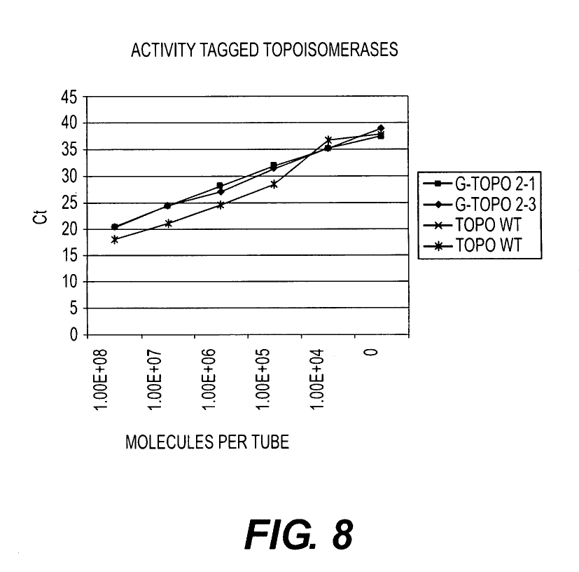

Protein-G tagged vaccinia topoisomerase was produced in BL21-(DE3) cells and affinity purified using the CBP tag. Wt-Toposiomerase was purified by conventional column purification. The assay was performed using the Hox probe using methods described above to determine if the Protein G fusion would interfere with the function of the enzyme. The CBP-protein-G displays topoisomerase was found to have a specific activity about one order of magnitude lower than for the wt-topoisomerase (see FIG. 8). This demonstrates that the expression of the topoisomerase as a fusion protein does not substantially interfere with the activity of the enzyme moiety.

### Example 3

### Topoisomerase-Based ELISA for Quantitative Detection of an Analyte

Wells of a polyvinylchloride microtiter plate are coated with equal amounts of a purified monoclonal antibody against an analyte. Wells are washed and blocked with a non-specific protein such as bovine serum albumin (BSA) in an appropriate buffer such as phosphate buffered saline (PBS). Samples that may contain the analyte are diluted serially in an appropriate buffer. Wells are washed to remove the blocking agent. Equal volumes of sample containing various dilutions of the original sample are placed in the prepared wells, preferably in duplicate or triplicate. For quantitative assays, a series of known analyte concentrations are added to a series of separate wells. The plate is incubated under appropriate conditions of temperature and humidity for an appropriate amount of time to allow binding of analyte present in the sample to the antibody. After incubation, wells are washed to remove any unbound analyte.

Wells are exposed to an analyte-specific binding agent that includes an antibody moiety as the analyte-specific binding moiety, and a topoisomerase moiety as the enzyme moiety. The antibody moiety binds to an epitope on the analyte that is distinct from the antibody that was used to coat the well. The microtiter plate is incubated under conditions of temperature and humidity for an appropriate amount of time to allow binding of analyte present in the sample to the antibody in the analyte-specific binding agent. The components of the analyte-specific binding agent are not attached to each other by a linking group that would result in non-specific binding to the well. The wells are washed to remove any unbound analyte-specific binding agent.

The bound analyte specific binding agent in the wells is contacted with two at least partially double stranded DNA duplexes in a vaccinia virus DNA topoisomerase I reaction mixture. One of the DNA duplexes has at least one blunt end with the 5′ end of one of the strands having the sequence of CCCTTN₆ (SEQ ID NO: 13) wherein each N is independently any nucleotide. The other double stranded DNA duplex has a 5′ overhang that is complementary to the N₆ sequence such that after topoisomerase cleavage, the two duplexes form a substrate for the topoisomerase activity of vaccinia virus that can join the one strand of the double stranded duplexes.

A portion of the topoisomerase reaction mixture is removed from the well and transferred to a reaction mixture for qPCR including two primers designed to allow for specific amplification of a product from the topoisomerase product. The reaction is monitored to determine threshold cycle (Cₜ) by detection of bound SYBR green. A standard curve is generated based on the samples from the wells containing known amounts of analyte. The Cₜs of the samples containing an unknown amount of the analyte are determined, and the amount of analyte present in the original sample is determined using the standard curve generated using samples with known concentrations of analyte.

### Example 4

### Heterogeneous VEGF ELISA Assay Using Topoisomerase Activity as a Readout

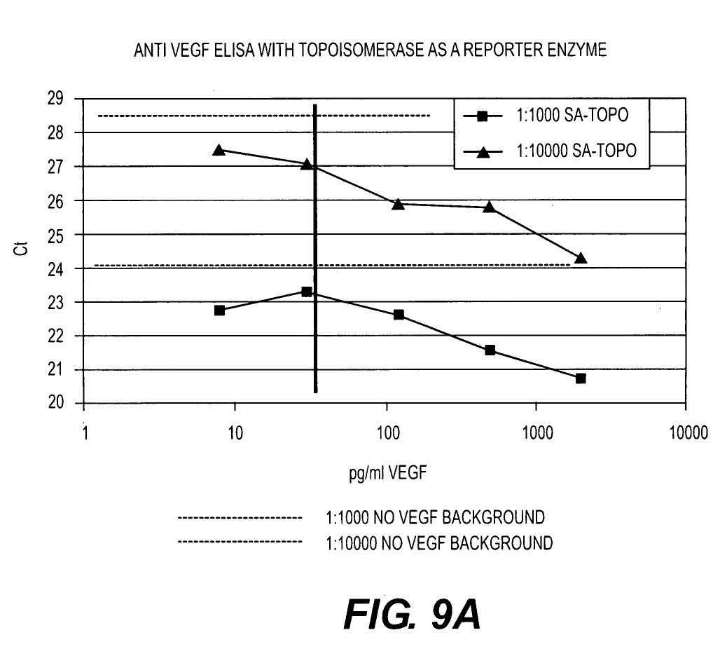

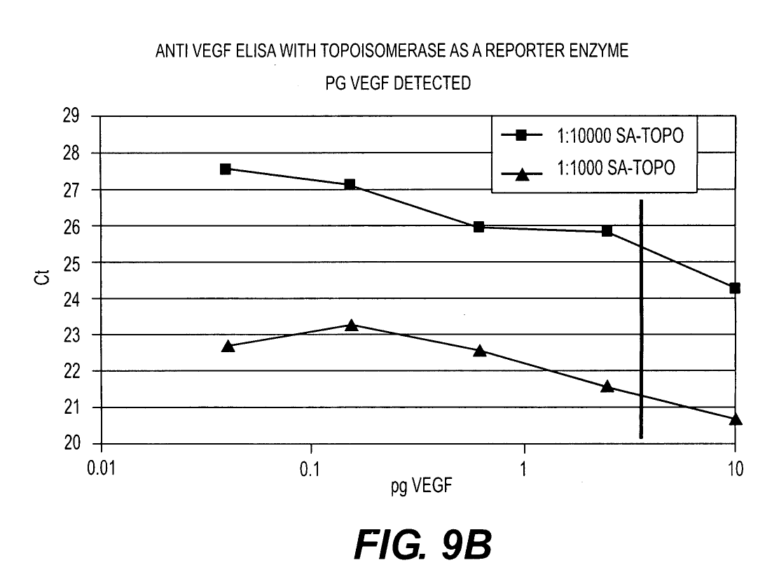

A VEGF ELISA was performed using topoisomerase activity as a readout for the presence of the analyte, VEGF. An ELISA kit for VEGF detection was purchased from a commercial supplier (R&D Systems). Binding and washing steps were performed per manufacturer's instructions, except for reducing the reaction volume from 100 ul to 5 ul, to the point of adding the detection reagent, the HRP-linked antibody. The SA-Topoisomerase detection reagent was added at a 1:1000 and 1; 10.000 dilution, and unbound detection reagent washed after the incubation. After 10 min incubation with 3 ul of the Topoisomerase substrates, 27 ul of PCR mixture were added and the PCR was performed as described in Example 1. The Cₜ values were plotted against the input concentration of VEGF (FIG. 9A). The dotted lines represent the assay background (no addition of VEGF). The grey bar represent the detection limit according to the manufacturer (30 pg/ml). In FIG. 9B the same data are represented as pg of VEGF detected. Again the grey bar represents the detection limit in the standard ELISA (3 pg per 100 ul). VEGF concentrations of 30 pg/ml could be reliably detected by the method. The absolute amount detected was about 150 fg or about 20-fold lower amounts than the commercial ELSA assay. These data demonstrate the sensitivity of the method of the invention relative to colorimetric enzymatic detection agents.

### Example 5

### Activity of T3 and T7 DNA ligases as His fusion proteins in various buffers

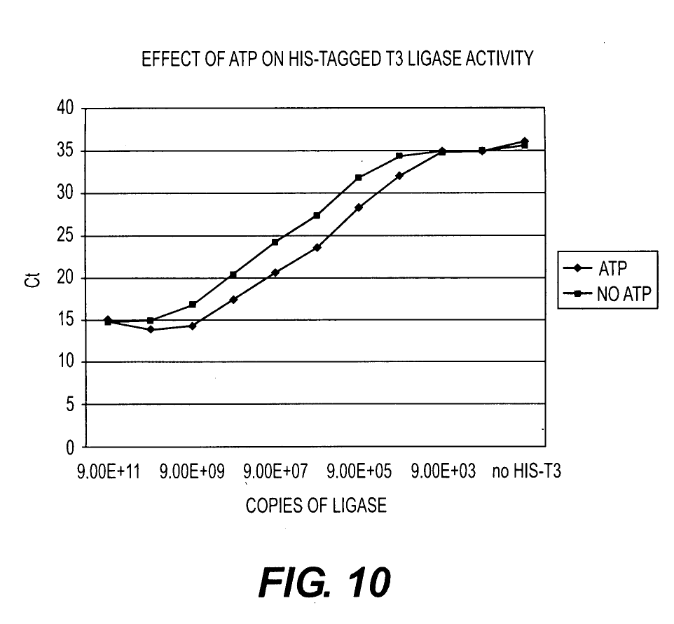

T3 and T7 DNA ligases were expressed as a recombinant His tagged protein to facilitate purification. The ligases were tested for activity in ligase buffer (50 mM Tris, pH 7.5, 7 mM MgCl₂, 1 mM DTT) in the presence or absence of 1 mM ATP. The generation of ligation product was determined by PCR amplification. The Cₜ for the amplification products for a range of ligase molecules is presented in FIG. 10. The data demonstrate both that the ligase does not have an absolute requirement for ATP and that the His tagged protein is functional.

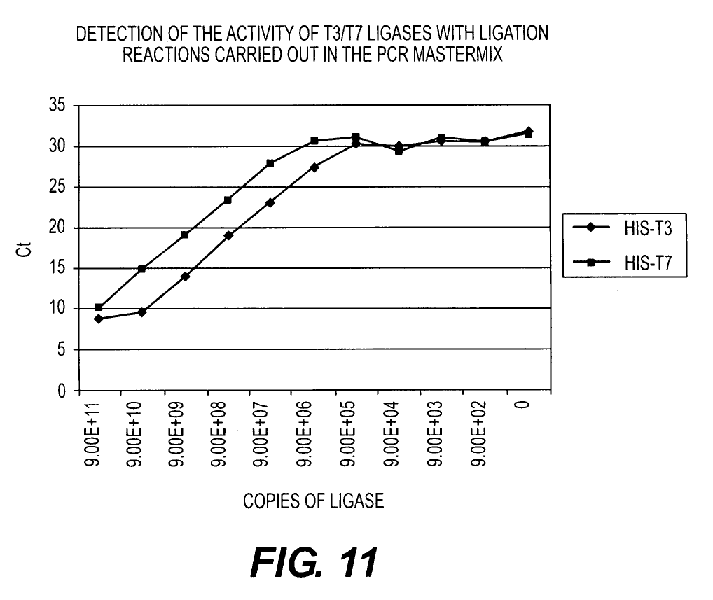

The T3 and T7 ligases were also tested in a PCR mastermix (Brilliant QPCR Mastermix, Stratagene, Catalog No. 600549). Both ligases as His tagged proteins were found to be functional in the PCR mastermix as determined by Cₜ (see FIG. 11).

It is noted that the T3 DNA ligase is both more effective than the T7 ligase, and that the T3 ligase is more active in the ligase buffer than the PCR mastermix.

T3 ligase has also been generated as a fusion protein with each streptavidin and Protein G. The fusion partner did not substantially interfere with the activity of the ligase as both ligases were found to be functional as fusion proteins.

### Example 6

### Ligase Based Detection Method to Screen for Protein-Protein Interactions

Random mutagenesis and high throughput screening methods can be used to screen for mutations that alter protein-protein interactions. A protein to be tested for interaction with its binding partner is subjected to random mutagenesis and subcloned into an expression vector containing an epitope tag. Individual colonies are picked and grown in culture. A portion of the each of the cultures is used to prepare a lysate containing the proteins from the randomly mutagenized library.

Wells of a polyvinylchloride microtiter plate are coated with equal amounts of a purified monoclonal antibody against the epitope tag expressed by each of the library members. Wells are washed and blocked with BSA in PBS. Extracts prepared from the individual library members are diluted in an appropriate buffer. Wells are washed to remove the blocking agent. Equal volumes of the diluted extract are placed in prepared wells, preferably in duplicate or triplicate. Positive control wells containing a version of the protein used for the mutagenesis known to bind the binding partner, and negative control wells not containing the protein or containing a version of the protein known to not interact with the binding partner are also prepared. The plate is incubated under appropriate conditions of temperature and humidity for an appropriate amount of time to allow binding of the library members in the extract to the antibody. After incubation, wells are washed to remove any unbound library members.

An appropriate analyte-specific binding agent including the binding partner attached to a ligase domain. The binding partner is generated as a fusion protein with a ligase domain using recombinant polypeptide methods such as those set forth above. The binding partner and the ligase domain are separated by a short, flexible protein sequence to reduce any effects that one domain may have on the other.

Wells are exposed to the binding partner-ligase fusion protein under conditions of temperature and humidity for an appropriate amount of time to allow binding of the library members to the binding partner. The wells are washed to remove any unbound analyte-specific binding agent.

The bound analyte specific binding agent in the wells is contacted with two double stranded DNA duplexes having compatible ends and at least one 5′-phosphate to allow for ligation of at least one strand to form a template for PCR. The two duplexes act as a substrate for a ligase under conditions to permit ligation. The ligation mixture is incubated at the appropriate temperature for a defined period of time.

A portion of the ligation reaction mixture is removed from the well and transferred to a reaction mixture for PCR including two primers designed to allow for specific amplification of a product from the ligation reaction. At the end of the amplification reaction, a portion of the reaction mixture is removed and subject to gel electrophoresis and staining with ethidium bromide to detect the presence of an amplification product indicating the interaction between the library member and the binding partner. Library members having the desired characteristics are further analyzed.

### EXEMPLARY EMBODIMENTS

1. A method for detection of an analyte in a sample comprising:

(a) incubating an analyte-specific binding agent with the analyte under conditions to permit binding, wherein the analyte-specific binding agent comprises an analyte-specific binding molecule attached to a ligase;

(b) incubating the bound analyte-specific binding agent of (a) with a first double stranded nucleic acid molecule and a second nucleic double stranded acid molecule in a reaction mixture, wherein the first nucleic acid molecule is ligated to the second nucleic acid molecule in the presence of the ligase;

(c) incubating at least a portion of the reaction mixture of (b) with an amplification reaction mixture comprising a first oligonucleotide primer, a second oligonucleotide primer, a DNA polymerase, and at least one dNTP, wherein the first oligonucleotide primer specifically binds the first nucleic acid molecule and the second oligonucleotide primer specifically binds the second nucleic acid molecule to permit formation of an amplification product; and incubating under conditions to permit nucleic acid amplification; and

(d) detecting an amplification product.

2. A method for detection of an analyte in solution comprising:

(a) incubating an analyte-specific binding agent with an analyte under conditions to permit binding, wherein the analyte-specific binding agent comprises an analyte-specific binding molecule attached to a ligase;

(b) incubating the bound analyte-specific binding agent of (a) with one or more polynucleotide substrate molecules, each having two ends in a reaction mixture, with the ligase, wherein the ligase joins two ends of the substrate molecules to generate a template for nucleic acid amplification;

(c) incubating at least a portion of the reaction mixture (b) with an amplification reaction;

(d) detecting an amplification product.

3. The method of embodiment 1 or 2, wherein the ligase is at least a catalytic portion of an enzyme selected from the group consisting of topoisomerase and DNA ligase.
4. The method of embodiment 3, wherein the ligase is a sequence-specific topoisomerase.
5. The method of embodiment 3, wherein the ligase is at least a catalytic portion of an enzyme selected from the group consisting of vaccinia virus DNA topoisomerase I and molluscum contagiosum virus (MCV) topoisomerase.
6. The method of embodiment 1 or 2, further comprising formation of a topoisomerase-nucleic acid bound intermediate.
7. The method of embodiment 3, wherein the ligase is at least a catalytic portion of an enzyme selected from the group consisting of T3 DNA ligase, T4 DNA ligase, and T7 DNA ligase.
8. The method of embodiment 1 or 2, wherein the analyte-specific binding molecule is selected from the group consisting of monoclonal antibody, polyclonal antibody, lectin, cell surface receptor, receptor ligand, peptide, carbohydrate, aptamer, biotin, streptavidin, avidin, protein A, and protein G; and any binding fragments thereof.
9. The method of embodiment 1 or 2, wherein the analyte is selected from the group consisting of protein, oligonucleotide, cell surface receptor, and receptor ligand.
10. The method of embodiment 1 or 2, wherein the analyte is bound to a solid support.
11. The method of embodiment 10, wherein the solid support is selected from the group consisting of polystyrene, derivatized polystyrene, a membrane, nitrocellulose, PVDF membrane, nylon membrane, latex bead, glass bead, silica bead, paramagnetic microsphere, latex microsphere, and microtiter well.
12. The method of embodiment 1 or 2, further comprising providing a third oligonucleotide that hybridizes to the first oligonucleotide primer, the second oligonucleotide primer, or both.
13. The method of embodiment 1 or 2, wherein the amplification product is a specific amplification product.
14. The method of embodiment 13, wherein the specific amplification product is determined by size.
15. The method of embodiment 1 or 2, wherein the detecting further comprises quantitation of the amplification product.
16. The method of embodiment 15, wherein the quantitation comprises quantitative PCR.
17. The method of embodiment 1, wherein the first double-stranded nucleic acid and the second double-stranded nucleic acid have compatible cohesive ends, or the method of embodiment of 2, wherein the ends in the reaction mixture have compatible cohesive ends.
18. The method of embodiment 1, wherein either the first double-stranded nucleic acid or the second double-stranded nucleic acid comprises a sequence-specific topoisomerase cleavage site, or the embodiment of method 2 wherein at least one of the ends comprises a sequence-specific topoisomerase cleavage site.
19. The method of embodiment 17, wherein at least one of the compatible cohesive ends is generated by sequence-specific topoisomerase cleavage.
20. The method of embodiment 18, wherein cleavage of the double-stranded nucleic acid results in formation of a double stranded portion of the nucleic acid that does not stably hybridize under conditions that permit ligation.
21. The method of embodiment 12, wherein the third oligonucleotide is selected from the group consisting of TaqMan® probe and Sentinel® Molecular Beacons probe.
22. The method of embodiment 1 or 2, wherein the first oligonucleotide primer comprises the sequence 5′-TCCACGGAGCTGTCTAGCG-3′ (SEQ ID NO: 10) and the second oligonucleotide primer comprises the sequence 5′-TGACGCCCGAAGCCAAGTG-3′ (SEQ ID NO: 11).
23. The method of embodiment 1 or 2, wherein the first double stranded nucleic acid molecule comprises:

(SEQ ID NO: 2)

5′-TGACGCCCGAAGCCAAGTGCGGGACGGCTTCTCCAGCTTGGCCCCTT
ATGGGT-3′

(SEQ ID NO: 3)

3′-ACTGCGGGCTTCGGTTCACGCCCTGCCGAAGAGGTCGAACCGGGGAA
TACCCTTGCT-5′

and the second double nucleic acid molecule comprises:

(SEQ ID NO: 4)

5′-ATGGGAACGAGCAGACCGACCGCTAGACAGCTCCGTGGA-3′

(SEQ ID NO: 5)

3′-CGTCTGGCTGGCGATCTGTCGAGGCACCT-5′.

24. The method of embodiment 1 or 2, wherein a nucleic acid of a non-template generating strand includes modifications.
25. The method of embodiment 1 or 2, wherein the DNA polymerase is a non-thermostable polymerase or a thermostable polymerase.
26. The method of embodiment 25, wherein the DNA polymerase is a non-thermostable polymerase selected from the group consisting of T3 DNA polymerase, T4 DNA polymerase, T5 DNA polymerase, T7 DNA polymerase, Klenow fragment, Φ29 DNA polymerase, and E. coli DNA polymerase I.
27. The method of embodiment 25, wherein the DNA polymerase is a thermostable polymerase selected from the group consisting of Pyrococcus furiosus (Pfu) DNA polymerase, Thermus thermophilus (Tth) DNA polymerase, Bacillus stearothermophilus DNA polymerase, Thermococcus litoralis (Tli) DNA polymerase, 9° Nm DNA polymerase, Thermotoga maritima (Tma) DNA polymerase, Thermus aquaticus (Taq) DNA polymerase, Pyrococcus kodakaraensis (KOD) DNA polymerase, JDF-3 DNA polymerase, and Pyrococcus GB-D (PGB-D) DNA polymerase.
28. The method of embodiment 1 or 2, further comprising removing unbound analyte specific binding agent before step (b).
29. A method for detection of an analyte in a sample comprising:

(a) incubating an analyte-specific binding agent with the analyte under conditions to permit binding, wherein the analyte-specific binding agent comprises an analyte-specific binding molecule attached to a reverse transcriptase;

(b) incubating the bound analyte-specific binding agent of (a) with an RNA molecule in a reaction mixture under conditions to permit reverse transcription of the RNA to generate a cDNA molecule;

(c) incubating at least a portion of the reaction mixture of (b) with an amplification reaction mixture comprising a first oligonucleotide primer, a second oligonucleotide primer, a DNA polymerase, and at least one dNTP, wherein the first oligonucleotide primer specifically binds the cDNA molecule and the second oligonucleotide primer specifically binds a complement of the cDNA molecule to permit formation of an amplification product; and incubating under conditions to permit nucleic acid amplification; and

(d) detecting an amplification product.

30. The method of embodiment 29, wherein the reverse transcriptase is selected from the group consisting of at least a catalytic portion of MMLV reverse transcriptase and AMV reverse transcriptase.
31. The method of embodiment 30, wherein the analyte-specific binding molecule is selected from the group consisting of monoclonal antibody, polyclonal antibody, lectin, cell surface receptor, receptor ligand, peptide, carbohydrate, aptamer, biotin, streptavidin, avidin, protein A, and protein G; and any binding fragments thereof.
32. The method of embodiment 29, wherein the analyte is selected from the group consisting of protein, oligonucleotide, cell surface receptor, hapten, small molecule, and receptor ligand.
33. The method of embodiment 29, wherein the analyte is bound to a solid support.
34. The method of embodiment 33, wherein the solid support is selected from the group consisting of polystyrene, derivatized polystyrene, a membrane, such as nitrocellulose, PVDF or nylon, a latex bead, a glass bead, a silica bead, paramagnetic or latex microsphere, microtiter well, paramagnetic microsphere, and a latex microsphere.
35. The method of embodiment 29, further comprising providing a third oligonucleotide that hybridizes to the first oligonucleotide, the second oligonucleotide, or both.
36. The method of embodiment 29, wherein the amplification product is a specific amplification product.
37. The method of embodiment 36, wherein the specific amplification product is determined by size.
38. The method of embodiment 29, wherein detection further comprises quantitation of the amplification product.
39. The method of embodiment 38, wherein quantitation comprises quantitative PCR.
40. The method of embodiment 29, wherein the RNA molecule has the sequence 5′-

(SEQ ID NO: 9)

5′GAUUGGAGCUCCACCGCGGUGGCGGCCGCUCUAGAACUAGUGGAUCCC
CCGGGCUGCAGGAAUCGAUAUCAAGCUAUCGAUACCGUCGACCUCGAGGG
GGGGCCGGUACCCCAGCUUUUGUUCCCUUUAGTGAGGGUUAAUUGCGCGC
UUGGCGUAAUCAUGGUCAUAGCUGUUUCCUGUGUGAAAU-3′.

41. The method of embodiment 29, wherein the RNA molecule includes modifications.
42. The method of embodiment 29, wherein the DNA polymerase is a non-thermostable polymerase or a thermostable polymerase.
43. The method of embodiment 42, wherein the DNA polymerase is a non-thermostable selected from the group consisting of T3 DNA polymerase, T4 DNA polymerase, T5 DNA polymerase, T7 DNA polymerase, Klenow fragment, ΦDNA polymerase, and E. coli DNA polymerase I.
44. The method of embodiment 42, wherein the DNA polymerase is a thermostable polymerase selected from the group consisting of Pyrococcus furiosus (Pfu) DNA polymerase, Thermus thermophilus (Tth) DNA polymerase, Bacillus stearothermophilus DNA polymerase, Thermococcus litoralis (Tli) DNA polymerase, 9° Nm DNA polymerase, Thermotoga maritima (Tma) DNA polymerase, Thermus aquaticus (Taq) DNA polymerase, Pyrococcus kodakaraensis (KOD) DNA polymerase, JDF-3 DNA polymerase, and Pyrococcus GB-D (PGB-D) DNA polymerase.
45. The method of embodiment 29, further comprising removing unbound analyte-specific binding agent before step (b).
46. A kit for detecting an analyte comprising:

an analyte-specific binding agent comprising an analyte-specific binding molecule attached to an enzyme; and

one or more polynucleotide substrates for the enzyme;

wherein the activity of the enzyme on the one or more polynucleotides produces a PCR template indicative of the presence of said analyte, and packaging material therefore.

47. The kit of embodiment 46, wherein the analyte specific binding molecule is selected from the group consisting of monoclonal antibody, polyclonal antibody, lectin, cell surface receptor, receptor ligand, peptide, carbohydrate, aptamer, biotin, streptavidin, avidin, protein A, and protein G; and any binding fragments thereof.
48. The kit of embodiment 46, wherein the enzyme molecule is selected from the group consisting of topoisomerase, ligase, and reverse transcriptase.
49. The kit of embodiment 46, wherein the polynucleotide substrates comprise two DNA polynucleotide substrates wherein one of the substrates includes a topoisomerase cleavage recognition site and the enzyme moiety is a sequence specific topoisomerase.
50. The kit of embodiment 46, wherein the polynucleotides have compatible cohesive ends and the enzyme is ligase.
51. The kit of embodiment 46, wherein the polynucleotide comprises an RNA polynucleotide and the enzyme is a reverse transcriptase.
52. The kit of embodiment 46, further comprising a polymerase for amplification of a PCR product.
53. An analyte-specific binding agent comprising an analyte-specific binding molecule attached to an enzyme molecule.
54. The agent of embodiment 53, wherein the analyte specific binding moiety is selected from the group consisting of monoclonal antibody, polyclonal antibody, lectin, cell surface receptor, receptor ligand, peptide, carbohydrate, aptamer, biotin, streptavidin, avidin, protein A, and protein G; and any binding fragments thereof.
55. The agent of embodiment 53, wherein the enzyme is selected from the group consisting of topoisomerase, ligase, and reverse transcriptase.
56. A composition comprising an enzyme coupled to the antibody binding domain.
57. The composition of embodiment 56, wherein the antibody binding domain is selected from the group consisting of Protein A, Protein G, and Protein L.
58. The composition of embodiment 56, wherein the composition is coupled to an anti-immunoglobulin antibody.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for detection of an analyte in a sample comprising:
(a) incubating an analyte-specific binding agent with the analyte under conditions to permit binding, wherein the analyte-specific binding agent comprises an analyte-specific binding molecule attached to a ligase;
(b) incubating the bound analyte-specific binding agent of (a) with a first double stranded nucleic acid molecule and a second nucleic double stranded acid molecule in a reaction mixture, wherein the first nucleic acid molecule is ligated to the second nucleic acid molecule in the presence of the ligase;
(c) incubating at least a portion of the reaction mixture of (b) with an amplification reaction mixture comprising a first oligonucleotide primer, a second oligonucleotide primer, a DNA polymerase, and at least one dNTP, wherein the first oligonucleotide primer specifically binds the first nucleic acid molecule and the second oligonucleotide primer specifically binds the second nucleic acid molecule to permit formation of an amplification product; and incubating under conditions to permit nucleic acid amplification; and
(d) detecting an amplification product.

2. The method of claim 1, wherein the ligase is at least a catalytic portion of an enzyme selected from the group consisting of topoisomerase and DNA ligase.

3. The method of claim 1, wherein the ligase is a sequence-specific topoisomerase.

4. The method of claim 1, wherein the ligase is at least a catalytic portion of an enzyme selected from the group consisting of vaccinia virus DNA topoisomerase I and molluscum contagiosum virus (MCV) topoisomerase.

5. The method of claim 1, wherein the ligase is at least a catalytic portion of an enzyme selected from the group consisting of T3 DNA ligase, T4 DNA ligase, T5 DNA ligase, Klenow, E. coli DNA polymerase, Φ29 DNA polymerase, and T7 DNA ligase.

6. The method of claim 1, wherein the analyte is bound to a solid support.

7. The method of claim 1, further comprising providing a third oligonucleotide that hybridizes to the first oligonucleotide primer, the second oligonucleotide primer, or both.

8. The method of claim 1, wherein the detecting further comprises quantitation of the amplification product.

9. The method of claim 7, wherein the third oligonucleotide is selected from the group consisting of TaqMan® probe and Sentinel® Molecular Beacons probe.

10. The method of claim 1, wherein the first oligonucleotide primer comprises the sequence 5′-TCCACGGAGCTGTCTAGCG-3′ (SEQ ID NO: 10) and the second oligonucleotide primer comprises the sequence 5′-TGACGCCCGAAGCCAAGTG-3′ (SEQ ID NO: 11).

11. The method of claim 1, wherein the first double stranded nucleic acid molecule comprises:
(SEQ ID NO: 2)
5′-TGACGCCCGAAGCCAAGTGCGGGACGGCTTCTCCAGCTTGGCCCCTT
ATGGGT-3′
(SEQ ID NO: 3)
3′-ACTGCGGGCTTCGGTTCACGCCCTGCCGAAGAGGTCGAACCGGGGAA
TACCCTTGCT-5′
and the second double nucleic acid molecule comprises:
(SEQ ID NO: 4)
5′-ATGGGAACGAGCAGACCGACCGCTAGACAGCTCCGTGGA-3′
(SEQ ID NO: 5)
3′-CGTCTGGCTGGCGATCTGTCGAGGCACCT-5′.

12. The method of claim 25, wherein the DNA polymerase is a non-thermostable polymerase selected from the group consisting of T3 DNA polymerase, T4 DNA polymerase, T5 DNA polymerase, T7 DNA polymerase, Klenow fragment, Φ29 DNA polymerase, and E. coli DNA polymerase I.

13. The method of claim 25, wherein the DNA polymerase is a thermostable polymerase selected from the group consisting of Pyrococcus furiosus (Pfu) DNA polymerase, Thermus thermophilus (Tth) DNA polymerase, Bacillus stearothermophilus DNA polymerase, Thermococcus litoralis (Tli) DNA polymerase, 9° Nm DNA polymerase, Thermotoga maritima (Tma) DNA polymerase, Thermus aquaticus (Taq) DNA polymerase, Pyrococcus kodakaraensis (KOD) DNA polymerase, JDF-3 DNA polymerase, and Pyrococcus GB-D (PGB-D) DNA polymerase.

14. The method of claim 1, further comprising removing unbound analyte specific binding agent before step (b).

15. A method for detection of an analyte in a sample comprising:
(a) incubating an analyte-specific binding agent with the analyte under conditions to permit binding, wherein the analyte-specific binding agent comprises an analyte-specific binding molecule attached to a reverse transcriptase;
(b) incubating the bound analyte-specific binding agent of (a) with an RNA molecule in a reaction mixture under conditions to permit reverse transcription of the RNA to generate a cDNA molecule;
(c) incubating at least a portion of the reaction mixture of (b) with an amplification reaction mixture comprising a first oligonucleotide primer, a second oligonucleotide primer, a DNA polymerase, and at least one dNTP, wherein the first oligonucleotide primer specifically binds the cDNA molecule and the second oligonucleotide primer specifically binds a complement of the cDNA molecule to permit formation of an amplification product; and incubating under conditions to permit nucleic acid amplification; and
(d) detecting an amplification product.

16. The method of claim 15, wherein the reverse transcriptase is at least a catalytic portion of an enzyme selected from the group consisting of MMLV reverse transcriptase and AMV reverse transcriptase.

17. The method of claim 15, wherein the RNA molecule has the sequence 5′-
(SEQ ID NO: 9)
5′GAUUGGAGCUCCACCGCGGUGGCGGCCGCUCUAGAACUAGUGGAUCCC
CCGGGCUGCAGGAAUCGAUAUCAAGCUAUCGAUACCGUCGACCUCGAGGG
GGGGCCGGUACCCCAGCUUUUGUUCCCUUUAGTGAGGGUUAAUUGCGCGC
UUGGCGUAAUCAUGGUCAUAGCUGUUUCCUGUGUGAAAU-3′.

18. The method of claim 15, wherein the DNA polymerase is a non-thermostable selected from the group consisting of T3 DNA polymerase, T4 DNA polymerase, T5 DNA polymerase, T7 DNA polymerase, Φ29 DNA polymerase, Klenow fragment, and E. coli DNA polymerase I.

19. The method of claim 15, wherein the DNA polymerase is a thermostable polymerase selected from the group consisting of Pyrococcus furiosus (Pfu) DNA polymerase, Thermus thermophilus (Tth) DNA polymerase, Bacillus stearothermophilus DNA polymerase, Thermococcus litoralis (Tli) DNA polymerase, 9° Nm DNA polymerase, Thermotoga maritima (Tma) DNA polymerase, Thermus aquaticus (Taq) DNA polymerase, Pyrococcus kodakaraensis (KOD) DNA polymerase, JDF-3 DNA polymerase, and Pyrococcus GB-D (PGB-D) DNA polymerase.

20. A kit for detecting an analyte comprising:
an analyte-specific binding agent comprising an analyte-specific binding molecule attached to an enzyme; and
one or more polynucleotide substrates for the enzyme;
wherein the activity of the enzyme on the one or more polynucleotides produces a PCR template indicative of the presence of said analyte,
and packaging material therefore.
